(19) United States
(12) Patent Application Publication
(10) Pub. No.: US 2011/0053991 A1
(43) Pub. Date: Mar. 3, 2011

(54) Treatment of Histone Deacetylase Mediated Disorders

(76) Inventors: Lia Gore, Denver, CO (US); Deborah De Ryckere, Boulder, CO (US)

(21) Appl. No.: 12/743,809

(22) PCT Filed: Nov. 19, 2008
(86) PCT No.: PCT/US08/84072

§ 371 Date: Nov. 11, 2010

Related U.S. Application Data
(60) Provisional application No. 60/989,053, filed on Nov. 19, 2007.

(51) Int. Cl. A61K 31/4406; A61P 35/00

(52) U.S. Cl. 514/357

(57) ABSTRACT

Provided herein are pharmaceutical agents, pharmaceutical compositions, methods of treatment, treatment regimens and kits for the treatment of histone deacetylase mediated disorders.

### CROSS-REFERENCES TO RELATED APPLICATIONS

This application claims priority to U.S. Provisional Application No. 60/989,053, filed Nov. 19, 2007, which is herein incorporated by reference in its entirety for all purposes.

### BACKGROUND OF THE INVENTION

Histone deacetylation is a characteristic feature of cancer cells. Histones are small proteins that are tightly complexed with DNA to form a nucleosome, which is further connected by linker DNA to form a solenoid. Histones extending from the nucleosomal core are enzymatically modified, affecting chromatin structure and gene expression. Specifically, histones are modified by histone deacetylases (HDACs) by removing an acetyl group. The inhibition of HDACs is associated with cell cycle arrest (as well as increased differentiation and apoptosis and the inhibition of proliferation, angiogenesis and metastasis). In order to survive, a cell must pass through the cell cycle, which has four distinct phases: G1, S, G2 and M.

There is a continuing need for methods and compositions for treating histone deacetylase mediated disorders, such as cancer. Disclosed herein are methods, compositions and kits that meet these and other needs in the art.

### SUMMARY OF THE INVENTION

Certain embodiments of the present invention provide methods, compositions and kits for treating histone deacetylase mediated disorders, such as cancer. Methods include administering to a patient a first amount of a Class I selective HDAC inhibitor and a second amount of a second HDAC inhibitor.

In one aspect, a method for treating histone deacetylase mediated disorders, such as cancer, in a subject in need thereof is provided. The method includes administering to the patient a first amount of a Class I selective HDAC inhibitor and a second amount of a second HDAC inhibitor.

In another aspect, a pharmaceutical composition for treating histone deacetylase mediated disorders, such as cancer, is provided. The pharmaceutical composition includes a first amount of a Class I selective HDAC inhibitor and a second amount of a second HDAC inhibitor.

In another aspect, kit for treating histone deacetylase mediated disorders, such as cancer, is provided. The kit includes a first pharmaceutical composition comprising a first amount of a Class I selective HDAC inhibitor and a second pharmaceutical composition comprising a second amount of a second HDAC inhibitor.

### BRIEF DESCRIPTION OF THE DRAWINGS

FIG. 1 illustrates the anti-tumor activity of vorinostat and entinostat toward acute leukemia cell lines. Cultures of the indicated ALL (top panels) and AML (bottom panels) cell lines were exposed to various concentrations of vorinostat (left panels) or entinostat (right panels) for 48 hours. A) The number of cells relative to cultures treated with vehicle only (DMSO) was determined by MTT assay. Mean values and standard errors were derived from 4-7 independent experiments. B) The number of proliferating cells relative to cultures treated with vehicle only was determined by measurement of BrdU incorporation using an ELISA. Mean values and standard errors were derived from 3-4 independent experiments.

FIG. 2 illustrates induction of dose-dependent cell death of mixed lineage leukemia cell lines by vorinostat and entinostat. Cultures of the indicated cell lines were exposed to vorinostat, entinostat, or vehicle only (DMSO) for 48 hours. Apoptotic and dead cells were identified by flow cytometric analysis of cells stained with propidium iodide and YO-PRO®-1. Cells undergoing apoptosis are stained with YO-PRO®-1, but are impermeable to propidium iodide. Dead cells and cells in late apoptosis are permeable to both dyes. Viable cells are not stained by either dye. Mean (+/−SEM) percentages of apoptotic and dead cells were derived from 3-7 independent experiments.

FIG. 3 illustrates the accumulation of mixed lineage leukemia cell lines in different phases of the cell cycle following treatment with vorinostat or entinostat. Cultures of the indicated cell lines were exposed to vorinostat, entinostat, or vehicle only (DMSO) for 48 hours. DNA content was determined by flow cytometric analysis of propidium iodide stained cells. The fractions of cells with G1, S, and G2/M phase DNA contents were determined. Mean values and standard errors were derived from 4-9 independent experiments.

FIG. 4 illustrates the mediation of differential changes in expression of differentiation markers on pediatric acute leukemia cell lines by vorinostat and entinostat. Mixed lineage AML (Molm14), mixed lineage ALL (RS411), B cell ALL (REH), and T cell ALL (CEM) cell lines were treated with the indicated concentrations of vorinostat or entinostat or with vehicle only (DMSO) for 72 hrs. Expression of differentiation markers was determined by flow cytometric analysis of cells stained with fluorochrome-labeled antibodies that recognize A) CD11b, B) CD13, C) HLA-DR, D) CD33, E) Terminal deoxynucleotidyl transferase (TdT), or F) CD3, CD28. Mean values+/−SEM from 3-7 independent experiments are shown. Statistically significant differences in expression levels compared to DMSO treated controls were determined using the student's unpaired t-test and are indicated by * (p value<0.05) or ** (p value<0.01).

FIG. 5 illustrates the similarity of induction of cell death and changes in cell cycle and expression of differentiation markers between trichostatin A (TSA) and sodium phenyl butyrate (SPB) and induction by vorinostat and entinostat, respectively. Cultures of the indicated cell lines were exposed to trichostatin A, sodium phenyl butyrate, or vehicle only (DMSO) for 48 (A and B) or 72 (C) hours. A) Apoptotic and dead cells were identified as described in FIG. 2. Mean values and standard errors derived from 1-5 independent experiments are shown. B) The fractions of cells with G1, S, and G2/M phase DNA contents were determined as described in FIG. 3. Mean values and standard errors were derived from 3-5 independent experiments. C) The fraction of viable cells expressing the indicated differentiation markers was determined as described in FIG. 4. Mean values+/−SEM from 2 independent experiments are shown.

FIG. 6 illustrates synergistic anti-tumor activity exhibited by the combination of vorinostat (SAHA) and entinostat (SNDX-275) in the Nalm-6 B cell ALL cell line. Cultures of the indicated cell lines were exposed to low, medium, and high concentrations of vorinostat, entinostat, or both for 48 hours. Relative cell numbers were determined by MTT assay and interactions were determined by Median Effect Analysis. The two left panels show Combination Indeces (CI Values) at different effective concentrations of HDACIs in two independent experiments. CI Values less than 1 indicate a synergistic interaction. The right panel shows mean CI values and standard errors for each experiment.

FIG. 7 illustrates sequence-dependent synergistic anti-tumor activity exhibited by the combination of vorinostat (SAHA) and entinostat (SNDX-275) in the REH ALL and Molm14 AML cell lines. Cultures of the indicated cell lines were exposed to low, medium, and high concentrations of vorinostat, entinostat, or both. Cells were treated concurrently with both HDACIs for 48 hours or were treated with one HDACI for 24 hours, after which the first HDACI was removed and the cells were cultured with the second HDACI for an additional 48 hours. Relative cell numbers were determined by MTT assay and interactions were determined by Median Effect Analysis. Mean CI values and standard errors are shown. CI Values less than 1 indicate a synergistic reduction in viable cell number.

FIG. 8 illustrates the mean IC₅₀ values of vorinostat (SAHA) and entinostat (MS-275) against various cancer cell lines (also shown in Table 1).

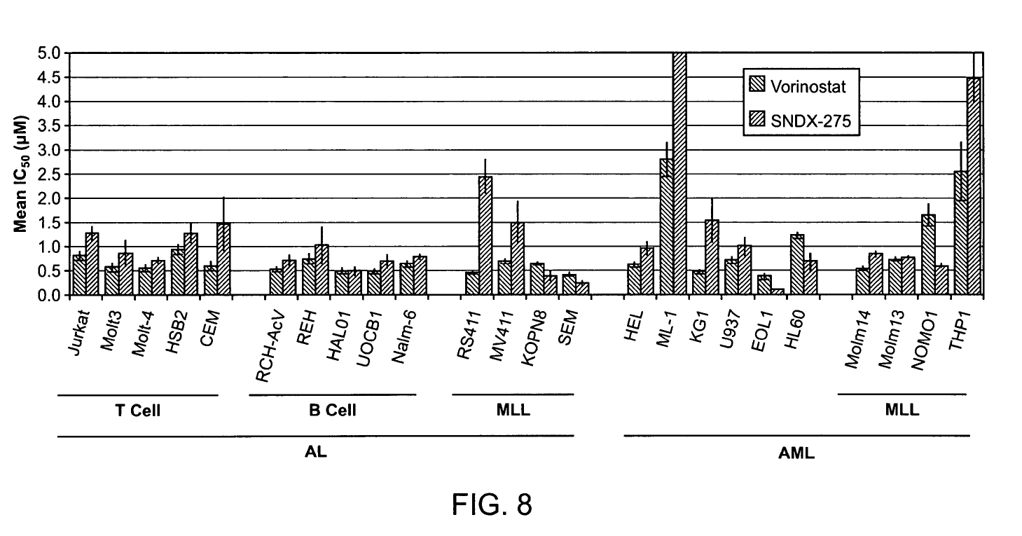

### DESCRIPTION OF THE INVENTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### CERTAIN TERMINOLOGY

Unless defined otherwise, all technical and scientific terms used herein have the same meaning as is commonly understood by one of skill in the art to which the claimed subject matter belongs. In the event that there is a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. It should also be noted that use of “or” means “and/or” unless stated otherwise. Furthermore, use of the term “including” as well as other forms, such as “include,” “includes,” and “included” is not limiting.

The HDACs are a family including at least eighteen enzymes, grouped in three classes (Class I, II and III). Class I HDACs include, but are not limited to, HADCs 1, 2, 3, 8 and 11. Class I HDACs can be found in the nucleus and are believed to be involved with transcriptional control repressors. Class II HDACs include, but are not limited to, HDACs 4, 5, 6, 7, and 9 and can be found in both the cytoplasm as well as the nucleus. Class III HDACs are believed to be NAD dependent proteins and include, but are not limited to, members of the Sirtuin family of proteins. Non-limiting examples of sirtuin proteins include SIRT1-7. As used herein, the term “selective HDAC” refers to an HDAC inhibitor that does not substantially interact with all three HDAC classes. As used herein, the term “Class I selective HDAC” refers to an HDAC inhibitor that does not substantially interact with a Class II HDAC or Class III HDAC.

The term “subject,” “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

Where combination treatments are contemplated, it is not intended that the agents described herein be limited by the particular nature of the combination. For example, the agents described herein may be administered in combination as simple mixtures as well as a chemical hybrids. An example of the latter is where the agent is covalently linked to a targeting carrier or to an active pharmaceutical. Covalent binding can be accomplished in many ways, such as, though not limited to, the use of a commercially available cross-linking agent.

As used herein, the terms “pharmaceutical combination,” “administering an additional therapy,” “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the first agents described herein, and at least one second agent, are both administered to a patient simultaneously in the form of a single entity or dosage. The term “non-fixed combination” means that at least one of the first agents described herein, and at least one second agent, are administered to a patient as separate entities either simultaneously, concurrently or sequentially with variable intervening time limits, wherein such administration provides effective levels of the two or more agents in the body of the patient. These also apply to cocktail therapies, e.g., the administration of three or more active ingredients.

As used herein, the terms “co-administration,” “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments the agents described herein will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the agents described herein and the other agent(s) are administered in a single composition. In some embodiments, the agents described herein and the other agent(s) are admixed in the composition.

The terms “effective amount,” “therapeutically effective amount” or “pharmaceutically effective amount” as used herein, refer to a sufficient amount of at least one agent being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising an agent as set forth herein required to provide a clinically significant decrease in a disease. An appropriate “effective” amount in any individual case may be determined using techniques, such as a dose escalation study.

The terms “administer,” “administering,” “administration,” and the like, as used herein, refer to the methods that may be used to enable delivery of agents or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the agents and methods described herein, e.g., as discussed in Goodman and Gilman, The Pharmacological Basis of Therapeutics (current ed.); Pergamon and Remington's, Pharmaceutical Sciences, Mack Publishing Co., Easton, Pa. (current ed.) In certain embodiments, the agents and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the agents described herein, and is relatively nontoxic, i.e., the material may be administered to an individual without causing undesirable biological effects or interacting in a deleterious manner with any of the components of the composition in which it is contained.

The term “carrier” as used herein, refers to relatively nontoxic chemical agent or agents that facilitate the incorporation of an agent into cells or tissues.

The term “pharmaceutically acceptable derivative or prodrug” as used herein, refers to any pharmaceutically acceptable salt, ester, salt of an ester or other derivative of an agent, which, upon administration to a recipient, is capable of providing, either directly or indirectly, a agent of this invention or a pharmaceutically active metabolite or residue thereof. Particularly favored derivatives or prodrugs are those that increase the bioavailability of the agents of this invention when such agents are administered to a patient (e.g., by allowing an orally administered agent to be more readily absorbed into blood) or which enhance delivery of the parent agent to a biological compartment (e.g., the brain or lymphatic system).

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

As used herein, the terms “cancer treatment” “cancer therapy” and the like encompasses treatments such as surgery, radiation therapy, administration of chemotherapeutic agents and combinations of any two or all of these methods. Combination treatments may occur sequentially or concurrently. Treatments(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

The term “disease” refers to any deviation from the normal health of a mammal and includes a state when disease symptoms are present, as well as conditions in which a deviation (e.g., infection, gene mutation, genetic defect, etc.) has occurred, but symptoms are not yet manifested. According to the present invention, the methods disclosed herein are suitable for use in a patient that is a member of the Vertebrate class, Mammalia, including, without limitation, primates, livestock and domestic pets (e.g., a companion animal). Typically, a patient will be a human patient.

“Controlled release”, “sustained release”, “prolonged release” or “time release” delivery are equivalent terms that describe the type of active agent delivery that occurs when the active agent is released from a delivery vehicle at an ascertainable and manipulatable rate over a period of time, which is generally on the order of minutes, hours or days, typically ranging from about thirty minutes to about 3 days, rather than being dispersed immediately upon entry into the digestive tract or upon contact with gastric fluid. A controlled release rate can vary as a function of a multiplicity of factors. Factors influencing the rate of delivery in controlled release may include the particle size, composition, porosity, charge structure, and degree of hydration of the delivery vehicle and the active ingredient(s), the acidity of the environment (either internal or external to the delivery vehicle), and the solubility of the active agent in the physiological environment, i.e., the particular location along the digestive tract.

### Methods of Treatment

In one aspect, a method for treating a histone deacetylase mediated disorder, such as cancer, in a subject in need thereof is provided. The method includes administering to the patient a first amount of a Class I selective HDAC inhibitor and a second amount of a second HDAC inhibitor. In some embodiments, the first amount and the second amount are together a therapeutically effective amount for treating the histone deacetylase mediated disorder. In some embodiments, the first amount and the second amount are together a therapeutically effective amount for treating cancer.

In some embodiments the subject is a mammal, such as a human. In a more specific embodiment, the human is an adult human. In various embodiments, the adult human is more than about 12 years old, more than about 16 years old or more than about 18 years old. In other embodiments, the human is a human that is 17 years old or less (e.g. a pediatric human).

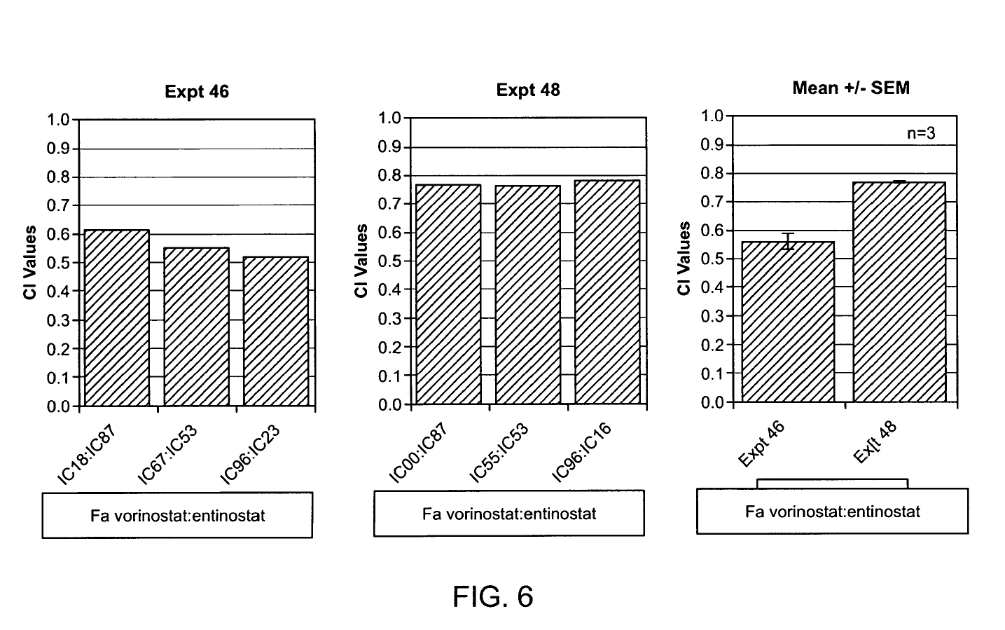

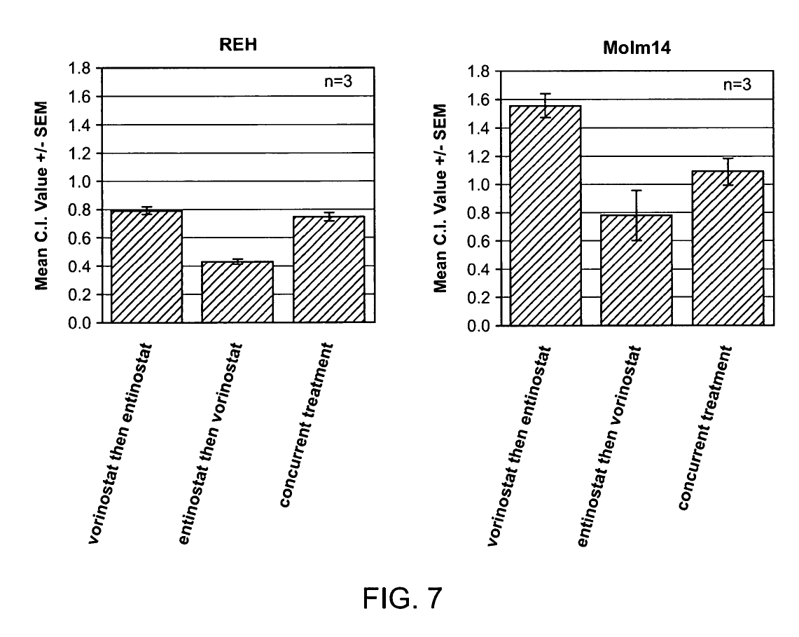

It has been found that, surprisingly, the combination of a Class I selective HDAC inhibitor (also referred to herein as the first agent) and a second HDAC inhibitor (also referred to herein as the second agent) exhibit a synergistic therapeutic anti-cancer effect. See, for example, FIGS. 6 and 7. Thus, in some embodiments, a first amount of the Class I selective HDAC inhibitor and the second amount of said second HDAC inhibitor are together an effective amount to provide a synergistic therapeutic anti-cancer effect. A “synergistic therapeutic anti-cancer effect,” as used herein, means that a given combination of at least 2 compounds exhibits synergy when tested in an anti-cancer assay (see Assays section below). Synergy may be assessed using the median-effect principle (Chou, et al., Adv Enzyme Regul 22:27-55 (1984)). This method is based on Michaelis-Menton kinetics and reduces combination effects to a numeric indicator, the combination index (C.I.). Where the combination index is less than 1, synergism is indicated. Where the combination index is equal to 1, summation (also commonly referred to as additivity) is indicated. Where the combination index is greater than 1, antagonism is indicated.

In certain embodiments, the first and/or second amounts are less than the minimum known amount to be therapeutically effective for the Class I selective HDAC inhibitor and/or second HDAC inhibitor, respectively. Thus, the second amount of the second HDAC inhibitor may be less than a therapeutic amount of the second HDAC inhibitor when the second HDAC inhibitor is administered in the absence of a Class I selective HDAC inhibitor. In other words, the second HDAC inhibitor may be administered in an amount that is less than would be a therapeutically effective amount when the second HDAC inhibitor is administered without the Class I selective HDAC inhibitor. Correspondingly, the first amount of the Class I selective HDAC inhibitor may be less than a therapeutic amount of the Class I selective HDAC inhibitor when the Class I selective HDAC inhibitor is administered in the absence of a second HDAC inhibitor. Thus, in some embodiments, the Class I selective HDAC inhibitor is administered in an amount that is less than would be a therapeutically effective amount when the Class I selective HDAC inhibitor is administered without the second HDAC inhibitor.

In some embodiments, the Class I selective HDAC inhibitor sensitizes the cancer cells to the second HDAC inhibitor. In some embodiments the second HDAC inhibitor sensitizes the cancer cells to a Class I selective HDAC inhibitor.

The Class I selective HDAC inhibitor inhibits (i.e. significantly and/or specifically decreases or arrests the activity of) at least one of HDAC-1, HDAC-2, HDAC-3, HDAC-8, or HDAC-11. In a specific embodiment, the first agent (i.e. the Class I specific HDAC inhibitor) inhibits HDAC-1. In another embodiment, the first agent inhibits HDAC-2. In yet another embodiment, the first agent inhibits HDAC-3. In still another embodiment, the first agent inhibits HDAC-11. In other embodiments, the first agent inhibits HDAC-1, HDAC-2, HDAC-3 and HDAC-11. In certain embodiments the first agent is entinostat (also commonly referred to as MS-275, SNDX-275, and N-(2-aminophenyl)-4-(N-(pyridin-3-ylmethoxycarbonyl)aminomethyl)benzamide), sodium phenyl butyrate, MGCD-0103 (N-(2-amino-phenyl)-4-[(4-pyridin-3-yl-pyrimidin-2-ylamino)-methyl]-benzamide), FK228, spiruchostatin A, SK7041, SK7068 or 6-amino nicotinamides. For clarity, the chemical structures of entinostat and MGCD-0103 are provided below.

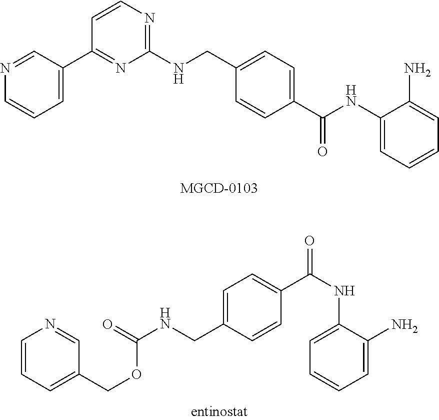

Another embodiment provided herein is a method wherein the Class I selective HDAC inhibitor is an agent that forces the arrest of a first cell cycle phase. In some embodiments, the first cell cycle phase that is arrested by the Class I selective HDAC inhibitor is G1. In certain embodiments, the HDAC inhibitor that forces G1 arrest is entinostat. In another non-limiting example, the HDAC inhibitor that forces G1 arrest is MGCD-0103.

In some embodiments, the Class I selective HDAC inhibitor alters the expression of at least one hematopoietic differentiation marker. In some embodiments, the Class I selective HDAC inhibitor alters the expression of at least one hematopoietic differentiation marker on an AML cell. In various embodiments, the Class I selective HDAC inhibitor alters the expression of at least one hematopoietic differentiation marker on an ALL cell. In specific embodiments, the Class I selective HDAC inhibitor that alters the expression of at least one hematopoietic differentiation marker is entinostat.

In certain embodiments, the second HDAC inhibitor is a non-selective HDAC inhibitor. In some embodiments, the second HDAC inhibitor is vorinostat (also commonly referred to as suberoylanilide hydroxamic acid, SAHA, or N′-hydroxy-N-phenyl-octanediamide, Zonlinza®), pyroxamide, CBHA, trichostatin A (TSA), trichostatin C, salicylihydroxamic acid (SBHA), azelaic bihydroxamic acid (ABHA), azelaic-1-hydroxamate-9-analide (AAHA), 6-(3-chlorophenylureido) carpoic hydroxamic acid (3Cl-UCHA), oxamflatin, A-161906, scriptaid, PXD-101, LAQ-824, CHAP, MW2796, LBH589 or MW2996, MW2976, any of the hydroxamic acids disclosed in U.S. Pat. Nos. 5,369,108, 5,932,616, 5,700,811, 6,087,367, 6,511,990 (each of which are incorporated by reference in their entirety), SAHA (NVP-LAQ-824, PXD-1-1), carboxylic acids such as butanoic, valproic, and 4-phenylbutanoic acids; benzamides and derivatives such as N-acetyldinaline and CI-994; epoxides and cyclic tetrapeptides such as trapoxins, depeudecin, depsipeptide (FK 228), FR225497, Apicidin cyclic tetrapeptide, Apicidin Ia, Apicidin Ib, Apicidin Ic, Apicidin IIa, Apicidin IIb; short-chain fatty acids and derivatives such as sodium butyrate, isovalerate, valerate. 4-phenylbutyrate (4-PBA), phenylbutyrate (PB) propionate, butyaramide, isobutyramide, phylacetate, 3-bromopropionate, tributyrin, valproic acid, Valproate, and Pivanex™; a cyclic tetrapeptide containing a 2-amino-8-oxo-9,10-epoxy-decanoyl moiety, and a cyclic peptide without the 2-amino-8-oxo-9,10-epoxy-decanoyl moiety; Savicol®; Baceca®; and LBH589. In another embodiment, the second HDAC inhibitor is vorinostat, pyroxamide, CBHA, trichostatin A (TSA), trichostatin C, salicylihydroxamic acid (SBHA), azelaic bihydroxamic acid (ABHA), azelaic-1-hydroxamate-9-analide (AAHA), 6-(3-chlorophenylureido) carpoic hydroxamic acid (3Cl-UCHA), oxamflatin, A-161906, scriptaid, PXD-101, LAQ-824, CHAP, MW2796, LBH589 or MW2996. In some embodiments, the second agent is vorinostat or trichostatin A. In other embodiments, the second agent is vorinostat. For clarity, the chemical structure of vorinostat is provided below.

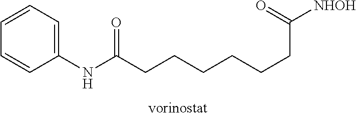

In some embodiments, the Class I selective HDAC inhibitor is entinostat or sodium phenyl butyrate and the second HDAC inhibitor is vorinostat or trichostatin A. In other embodiments, the Class I selective HDAC inhibitor is entinostat and the second HDAC inhibitor is vorinostat or trichostatin A. In certain embodiments, the Class I selective HDAC inhibitor is entinostat and the second HDAC inhibitor is vorinostat.

In another embodiment, the second HDAC inhibitor arrests a cell cycle phase. Where the Class I selective HDAC inhibitor arrests a cell cycle phase (e.g. at G₁) the second HDAC inhibitor arrests at a cell cycle phase that is different than the first cell cycle phase arrested by the Class I selective HDAC inhibitor. In certain embodiments of the present invention, the cell cycle phase arrested by the second HDAC inhibitor is G2 or S. In some embodiments, the second HDAC inhibitor that arrests G2 and/or S is vorinostat.

In some embodiments, the second HDAC inhibitor is an HDAC inhibitor that does not alter the expression of an hematopoietic differentiation marker. It is to be understood that an HDAC inhibitor that does not alter the expression of a hematopoietic differentiation marker may include an HDAC inhibitor that alters the expression of an hematopoietic differentiation marker to an insignificant extent (e.g., minimally). In certain embodiments, the Class I selective HDAC inhibitor is an HDAC inhibitor that alters the expression of at least one hematopoietic differentiation marker and the second HDAC inhibitor does not alter the expression of a hematopoietic differentiation marker.

In certain embodiments of the present invention, there is provided a method of treating a histone deacetylase mediated disorder by administering a first agent to a patient, wherein the first agent sensitizes the cancer to another agent, which is subsequently or simultaneously administered. In specific embodiments, the histone deacetylase mediated disorder that is sensitized by the first agent is cancer. In some embodiments, the first agent that sensitizes the histone deacetylase mediated disorder is an HDAC inhibitor that forces the arrest of G1. In some embodiments, the first agent is MS-275 and the second agent is SAHA. In other embodiments, the first agent is MS-275 and the second agent is a kinase inhibitor, as described herein above.

In some embodiments, the first agent (i.e. the Class I selective HDAC inhibitor) and the second agent (i.e. the second HDAC inhibitor) are administered sequentially. In certain embodiments, the first agent is administered to a patient first and the second agent is administered at a later time or date. In other embodiments, the first and second agents are administered simultaneously. In one embodiment, the first and second agents are administered simultaneously and the second agent is administered again, in the absence of the first agent, at a later time or date. In yet another embodiment, the first agent is administered, in the absence of the second agent, and the second agent is administered together with the first agent at a later time or date. In some embodiments, the first agent is administered as a first pharmaceutical composition and the second agent is administered as a second pharmaceutical composition (e.g. a kit). In other embodiments, the first and second agents are co-administered in a single pharmaceutical composition. In still other embodiments, the second agent is administered more slowly than the first agent. Specifically, in some embodiments where entinostat is given in combination with vorinostat (SAHA), dosing of entinostat precedes dosing of vorinostat by approximately 3, 6, 9, 12, 15, 18, 21, or 24 hours or more. In some embodiments, the dosing of entinostat precedes dosing of vorinostat by approximately 24 hours or more.

In certain embodiments, an additional therapeutic agent (also referred to herein as an additional agent) is administered to a patient first and at least one of the HDAC inhibitors is administered at a later time or date. In other embodiments, the additional agent and at least one of the HDAC inhibitors are administered simultaneously. In still another embodiment, the additional agent and at least one of the HDAC inhibitors are administered simultaneously and the additional agent is administered again, in the absence of the HDAC inhibitor, at a later time or date. In yet another embodiment, the additional agent is administered, in the absence of an HDAC inhibitor, and at least one of the HDAC inhibitors is administered together with the additional agent at a later time or date. In some embodiments, the additional agent is administered as a first pharmaceutical composition and at least one HDAC inhibitor is administered as a second pharmaceutical composition. In other embodiments, the additional agent and at least one of the HDAC inhibitors are co-administered in a single pharmaceutical composition. Additional agents are discussed below.

In some embodiments, the first amount is a therapeutically effective amount and the second amount is a therapeutically effective amount. In certain embodiments, the therapeutically effective amount of each agent (i.e. the Class I selective HDAC inhibitor and the second HDAC inhibitor) is, independently, about 0.01 to about 1,000 mg/m². In some embodiments, the therapeutically effective amount of each agent is, independently, from about 0.1 to about 500 mg/m². In other embodiments, the therapeutically effective amount of each agent is, independently, from about 0.5 to about 100 mg/m². In some embodiments wherein the first agent is entinostat, therapeutically effective amounts of the first agent are about 0.5 to about 15 mg/m². In other embodiments wherein the first agent is MS-275, therapeutically effective of the first agent amounts are about 2 to about 8 mg/m². In specific embodiments wherein the first agent is MS-275, the therapeutically effective amount of MS-275 is about 2, 4, 6 or 8 mg/m². In other embodiments wherein the first agent is MGCD-0103, therapeutically effective amounts of the first agent are about 5 to about 100 mg/m². In certain embodiments wherein the first agent is MGCD-0103, therapeutically effective amounts of the first agent are about 10 to about 80 mg/m². In other embodiments wherein the first agent is MGCD-0103, therapeutically effective amounts are about 12 to about 60 mg/m². In still other embodiments wherein the first agent is MGCD-0103, therapeutically effective amounts of the first agent are about 12.5 to about 36 mg/m². In specific embodiments wherein the first agent is MGCD-0103, the therapeutically effective amount of MGCD-0103 is about 12.5, 20, 27, 36, 40, 60 or 80 mg/m².

In certain embodiments, the first agent is administered in a regimen that is therapeutically effective. In various embodiments, the first agent is administered, by way of non-limiting example, three times a day, twice daily, once daily, five times a week, four times a week, three times a week, twice a week, once weekly, once every two weeks or once every 6 weeks. In certain embodiments, the second agent is administered in a regimen that is therapeutically effective. In various embodiments, the second agent is administered, by way of non-limiting example, three times a day, twice daily, once daily, five times a week, four times a week, three times a week, twice a week, once weekly, once every two weeks or once every 6 weeks. In certain embodiments, the additional agent is administered in a regimen that is therapeutically effective. In various embodiments, the additional agent is administered, by way of non-limiting example, three times a day, twice daily, once daily, five times a week, four times a week, three times a week, twice a week, once weekly, once every two weeks or once every 6 weeks.

In a specific embodiment, the first agent is administered on days 1 and 15 and the second agent is administered on days 3-5 and 10-12. In another specific embodiment, the first agent is administered on days 1, 8 and 15 and the second agent is administered on days 1-5 and 8-12 (i.e., on 5 consecutive days per week). In still another specific embodiment, the first agent is administered on days 1 and 15 and the second agent is administered on days 1-5 and 8-12 (i.e., on 5 consecutive days per week). In a specific embodiment, the first agent is administered on days 1 and 15 and the second agent is administered on days 3-5 and 10-12. In another specific embodiment, the first agent is administered on days 1, 8 and 15 and the second agent is administered daily. In still another specific embodiment, the first agent is administered on days 1 and 15 and the second agent is administered daily. In another specific embodiment, the first agent is administered on days 1 and 8 and the second agent is administered on days 2-5 and 9-11. In still other specific embodiments, the first agent is administered on days 1 and 8 and the second agent is administered, by way of non-limiting example, on days 2-8, 3-9, 2-15, 3-16 or 4-11.

In various embodiments, the histone deacetylase mediated disorder is, by way of non-limiting example, asthma, inflammatory bowel diseases such as Crohn's disease and ulcerative colitis, psoriasis, sarcoidois, and rheumatoid arthritis. In other embodiments, the histone deacetylase mediated disorder is an infection including, but not limited to, malaria, protozoal infections, EBV, HIV, hepatitis B and C, KSHV, toxoplasmosis and coccidiosis. In still other embodiments, the histone deacetylase mediated disorder is an autoimmune disorder including, but not limited to, conditions treatable by immune modulation, rheumatoid arthritis, autoimmune diabetes, lupus, multiple sclerosis, and allergies. In yet other embodiments, the histone deacetylase mediated disorder is a neurological disorder including, but not limited to, Huntington's disease, epilepsy, neuropathic pain, depression, and bipolar disorders. In other embodiments, the histone deacetylase mediated disorder is a proliferative disorder including, but not limited to, psoriasis, restenosis, autoimmune disease, proliferative responses associated with organ transplantation, and atherosclerosis. In still other embodiments, the histone deacetylase mediated disorder is a fibrogenic disorder including, but not limited to, scleroderma, keloid formation, pulmonary fibrosis and liver cirrhosis. In yet other embodiments, the histone deacetylase mediated disorder is a cardiac disorder including, but not limited to, cardiovascular conditions, cardiac hypertrophy, idiopathic cardiomyopathies, and heart failure. In some embodiments, the histone deacetylase mediated disorder is a hyperproliferative disorder including, but not limited to, hematologic and nonhematologic cancers, cancerous and precancerous skin lesions, leukemias, hyperplasias, fibrosis, angiogenesis, psoriasis, atherosclerosis, and smooth muscle proliferation in the blood vessels. In other embodiments, the histone deacetylase mediated disorder is a metabolic disease including, but not limited to, genetic related metabolic disorders, cystic fibrosis, peroxisome biogenesis disorder, alpha-1 anti-trypsin, adrenoleukodystrophy, and spinal muscular atrophy. In still other embodiments, the histone deacetylase mediated disorder is a malignant disease including, but not limited to, malignant fibrous histiocytoma, malignant mesothelioma, and malignant thymoma.

In some embodiments, the cancer treated by methods described herein is, by way of non-limiting example, brain cancer, breast cancer, lung cancer, ovarian cancer, pancreatic cancer, prostate cancer, renal cancer, colorectal cancer, leukemia, myeloid leukemia, acute myeloid leukemia (AML), glioblastoma, follicular lymphoma, pre-B acute leukemia, chronic lymphocytic B-leukemia, mesothelioma or small cell lung cancer. Additional cancers to be treated with the methods and compositions described herein include hematologic and non-hematologic cancers. Hematologic cancer includes multiple myeloma, leukemias, myelodysplastic syndromes, lymphomas, acute leukemia, acute lymphocytic leukemia (ALL), acute myeloid leukemia (AML)/acute nonlymphocytic leukemia (ANLL), chronic lymphocytic leukemia (CLL) and chronic myelogenous leukemia (CML). Lymphoma further includes Hodgkin's lymphoma and non-Hodgkin's lymphoma, cutaneous T-cell lymphoma (CTCL), pediatric acute leukemia, pediatric acute myeloid leukemia, pediatric acute lymphoid leukemia, juvenile myelomonocytic leukemia (JJMML/JCML), and mantle cell lymphoma (MCL). Non-hematologic cancer includes brain cancer, cancers of the head and neck, lung cancer, breast cancer, cancers of the reproductive system, cancers of the gastro-intestinal system, pancreatic cancer, and cancers of the urinary system, cancer of the upper digestive tract or colorectal cancer, bladder cancer or renal cell carcinoma, and prostate cancer.

In certain embodiments, the cancer is a pediatric cancer. In some embodiments, the pediatric cancer is selected from, by way of non-limiting example, brain cancer, leukemia, myeloid leukemia, acute myeloid leukemia (AML), glioblastoma, follicular lymphoma, pre-B acute leukemia, leukemias, myelodysplastic syndromes, lymphomas, acute leukemia, acute lymphocytic leukemia (ALL), acute myeloid leukemia (AML)/acute nonlymphocytic leukemia (ANLL), chronic myelogenous leukemia (CML), Hodgkin's lymphoma, non-Hodgkin's lymphoma, acute leukemia, acute lymphoid leukemia, juvenile myelomonocytic leukemia (JJMML/JCML), cancers of the reproductive system and cancers of the urinary system.

In some embodiments, the cancers to treat with the methods and compositions described herein include cancers that are epithelial malignancies (having epithelial origin), and any cancers (tumors) that express EGFR. Non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g., of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g., metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer. In some embodiments, the cancer is a pediatric cancer selected from dysplasias and xeroderma pigmentosum.

Additional histone deacetylase mediated cancers which are treated in various embodiments of the present invention include: cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. These cancers further include cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, metastatic melanoma, basal cell carcinoma, squamous cell carcinoma, other non-epithelial skin cancer; uterine or cervical cancer; uterine corpus cancer; ovarian, vulvar, vaginal, or other female genital cancers; prostate, testicular, penile or other male genital cancer; urinary bladder cancer; cancer of the kidney; renal, pelvic, or urethral cancer or other cancer of the genito-urinary organs; thyroid cancer or other endocrine cancer; chronic lymphocytic leukemia; and cutaneous T-cell lymphoma, both granulocytic and monocytic. In certain embodiments, the cancer is a pediatric cancer selected from, by way of non-limiting example, cancers of bones and joints, cancers of soft tissue, cancers of genital system, cancers of the eye and orbit, cancers of the nervous system, cancers of the lymphatic system, cancer of the liver, female genital cancers, cancer of the kidney, renal, pelvic or urethral cancer or other cancer of the genito-urinary organs, thyroid cancer or other endocrine cancer.

Yet other histone deacetylase mediated disorder cancers which may be treated using the compositions, combinations and methods described herein include: adenocarcinoma, angiosarcomas, astrocytoma, acoustic neuroma, anaplastic/high-grade astrocytoma, atypical teratoid/rhabdoid tumor, basal cell carcinoma, blastoglioma, chondrosarcoma, choriocarcinoma, chordoma, clear cell sarcoma of the kidney, clear cell sarcoma of the ovary, craniopharyngioma, cutaneous melanoma, cystadenocarcinoma, desmoplastic small round cell tumor, endotheliosarcoma, embryonal carcinoma, ependymoma, Ewing's family of tumors/peripheral neuroepithelioma, primitive neuroectodermal tumor (PNET), epithelial carcinoma, fibrosarcoma, ganglioglioma, ganglioneuroblastoma, gastric cancer, genitourinary tract cancers, germ cell tumors, non-germinomatous germ cell tumors, glioblastoma multiforme, hemangioblastoma, hepatoblastoma, hepatocellular carcinoma, hepatoma, histiocytosis syndromes, Kaposi's sarcoma, Langerhans cell histiocytosis, large cell carcinoma, leiomyosarcoma, liposarcoma, lymphangiosarcoma, lymphangioendotheliosarcoma, malignant fibrous histiocytoma, medullary thyroid carcinoma, medulloblastoma, meningioma mesothelioma, myelomas, myxosarcoma neuroblastoma, neuroblastoma, neurofibrosarcoma, ocular melanoma, oligodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdoid tumor of the kidney, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, small cell lung cancer and Wilm's tumor. In certain embodiments, the cancer is a pediatric cancer selected from, by way of non-limiting example, angiosarcoma, astrocytoma, acoustic neuroma, anaplastic/high-grade astrocytoma, atypical teratoid/rhabdoid tumor, chondrosarcoma, choriocarcinoma, chordoma, clear cell sarcoma of the kidney, clear cell sarcoma of the ovary, craniopharyngioma, desmoplastic small round cell tumor, endotheliosarcoma, embryonal carcinoma, ependymoma, Ewing's family of tumors/peripheral neuroepithelioma, primitive neuroectodermal tumor (PNET), fibrosarcoma, ganglioglioma, ganglioneuroblastoma, genitourinary tract cancers, germ cell tumors, non-germinomatous germ cell tumors, glioblastoma multiforme, hemangioblastoma, hepatoblastoma, hepatocellular carcinoma, histiocytosis syndromes, Langerhans cell histiocytosis, leiomyosarcoma, liposarcoma, lymphangiosarcoma, lymphangioendotheliosarcoma, malignant fibrous histiocytoma, medullary thyroid carcinoma, medulloblastoma, neuroblastoma, neurofibrosarcoma, oligodendroglioma, osteogenic sarcoma, retinoblastoma, rhabdoid tumor of the kidney, rhabdomyosarcoma, thyroid cancer, and Wilm's tumor.

### Pharmaceutical Compositions and Formulations

In another aspect, pharmaceutical compositions and formulations are provided for treating a histone deacetylase mediated disorder including a first amount of a Class I selective HDAC inhibitor and a second amount of a second HDAC inhibitor. In some embodiments, the histone deacetylase mediated disorder is cancer. The histone deacetylase mediated disorders, Class I selective HDAC inhibitors, second HDAC inhibitors, the first amount, the second amount, and cancers described above in the Method of Treatment section are equally applicable to the pharmaceutical compositions and formulations described herein.

Thus, in some embodiments, the pharmaceutical composition for treating cancer includes a first amount of a Class I selective HDAC inhibitor and a second amount of a second HDAC inhibitor. The pharmaceutical composition may also include a pharmaceutically acceptable excipient, carrier, buffer, adjuvant, and/or stabilizer. In some embodiments, the first amount and the second amount are together an effective amount to provide a synergistic therapeutic anti-cancer effect. The discussion above related to synergistic therapeutic anti-cancer effects are equally applicable to the pharmaceutical compositions and formulations described herein.

As described above, in some embodiments, the second amount of the second HDAC inhibitor is less than a therapeutic amount of the second HDAC inhibitor when the second HDAC inhibitor is administered without the Class I selective HDAC inhibitor. And as also described above, the first amount of the Class I selective HDAC inhibitor may be less than a therapeutic amount of the Class I selective HDAC when the Class I selective HDAC inhibitor is administered without the second HDAC inhibitor.

In some embodiments, the Class I selective HDAC inhibitor is entinostat, sodium phenyl butyrate, MGCD-0103, FK228, spiruchostatin A, SK7041, SK7068 or a 6 amino nicotinamide. The Class I selective HDAC inhibitor may also simply be entinostat. The second HDAC inhibitor may be vorinostat, pyroxamide, CBHA, trichostatin A, trichostatin C, salicylihydroxamic acid, azelaic bihydroxamic acid, azelaic-1-hydroxamate-9-analide, 6-(3-chlorophenylureido) carpoic hydroxamic acid (3Cl UCHA), oxamflatin, A 161906, scriptaid, PXD-101, LAQ-824, CHAP, MW2796, LBH589 or MW2996. In some embodiments, the second HDAC inhibitor is vorinostat or trichostatin C. In other embodiments, the second HDAC inhibitor is vorinostat.

In some embodiments the pharmaceutical composition is an oral dosage form or an injectable dosage form. The oral dosage from and/or injectable dosage forms may be configured to delay the release of the second HDAC inhibitor (e.g. vorinostat) relative to the Class I selective HDAC inhibitor (e.g. entinostat).

For example, in some embodiments, the pharmaceutical composition is an oral dosage form including a prolonged release portion. The prolonged release portion includes the second HDAC inhibitor (e.g. vorinostat). The pharmaceutical composition may further include an immediate release portion including the Class I selective HDAC inhibitor (e.g. entinostat).

Disclosures of oral dosage forms that swell to sizes that will prolong the residence time in the stomach are found in U.S. Pat. No. 5,007,790 (“Sustained-Release Oral Drug Dosage Form,” Shell, inventor; Apr. 16, 1991); U.S. Pat. No. 5,582,837 (“Alkyl-Substituted Cellulose-Based Sustained-Release Oral Drug Dosage Forms,” Shell, inventor; Dec. 10, 1996); U.S. Pat. No. 5,972,389 (“Gastric-Retentive Oral Drug Dosage Forms for the Controlled Release of Sparingly Soluble Drugs and Insoluble Matter,” Shell et al., inventors; Oct. 26, 1999); International (PCT) Patent Application WO 98/55107 (“Gastric-Retentive Oral Drug Dosage Forms for Controlled Release of Highly Soluble Drugs,” Shell et al., inventors; publication date Dec. 10, 1998); United States Patent Application Publication No. US 2001/0018707 A1 (“Extending the Duration of Drug Release Within the Stomach During the Fed Mode,” Shell et al., inventors, publication date Aug. 30, 2001); and International (PCT) Patent Application WO 96/26718 (“Controlled Release Tablet,” Kim, inventor: publication date Sep. 6, 1996). Each of the documents cited in this paragraph is incorporated herein in its entirety. In certain embodiments of the invention, the supporting matrix in the prolonged-release portion of the oral dosage form (e.g. a tablet or capsule) is a material that swells upon contact with gastrointestinal fluid to a size that is large enough to promote retention in the stomach.

In general, gastric-retentive (swellable) matrices contain binders that are water-swellable polymers, and suitable polymers are those that are non-toxic, that swell in a dimensionally unrestricted manner upon imbibition of water, and that release the drug gradually over time. Examples of polymers meeting this description are: cellulose polymers and substituted cellulose polymers, including alkyl-substituted, hydroxyalkyl-substituted, and carboxyalkyl-substituted celluloses, specific examples being, although not limited to, hydroxymethyl cellulose, hydroxyethyl cellulose, hydroxypropyl cellulose, hydroxypropylmethyl cellulose, and carboxymethylcellulose, and microcrystalline cellulose; polysaccharides and substituted polysaccharides (with substituents such as those listed above); poly(alkylene oxide)s; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane hydrogels; and crosslinked poly(acrylic acid)s and substituted crosslinked poly(acrylic acid)s (with substituents such as those listed above). Further examples are copolymers of the polymers listed above, including block copolymers and graft polymers. Specific examples of copolymers are PLURONIC® and TECTONIC®, which are polyethylene oxide-polypropylene oxide block copolymers available from BASF Corporation, Chemicals Div., Wyandotte, Mich., USA. Further examples are hydrolyzed starch polyacrylonitrile graft copolymers, commonly known as “Super Slurper” and available from Illinois Corn Growers Association, Bloomington, Ill., USA. Particularly preferred polymers are poly(ethylene oxide), hydroxypropyl methyl cellulose, and combinations of poly(ethylene oxide) and hydroxypropyl methyl cellulose.

The immediate-release portion of the dosage form may be a coating applied or deposited over the entire surface of a unitary prolonged-release core, or a single layer of a tablet constructed in two or more layers, one of the other layers being the prolonged-released portion. Immediate release of the drug from the immediate-release layer is achieved in any of a variety of ways. One example is by placing a drug in a layer or coating that is sufficiently thin to allow fast penetration by gastrointestinal fluid which then leaches the drug at a rapid rate. Another example is by incorporating the drug in a mixture that includes a supporting binder or other inert material that dissolves readily in gastrointestinal fluid, releasing the drug as the material dissolves. A third is the use of a supporting binder or other inert material that rapidly disintegrates into fine particles upon contact with gastrointestinal fluid, with both the binder particles and the drug quickly dispersing into the fluid. Examples of materials that rapidly disintegrate and disperse are lactose and microcrystalline cellulose. Hydroxypropyl methyl cellulose is a component that can serve both as a suspending agent and as a binder.

The therapeutic agents of the present invention may each be administered individually (e.g. a kit as set forth below) or in combination. When the two or more agents are administered in combination, they are administered without any additional components or with additional components in a pharmaceutical composition (e.g. a pharmaceutically acceptable excipient). In certain embodiments, the pharmaceutical compositions are prepared by admixing at least one active ingredient together with one or more carriers, excipients, buffers, adjuvants, stabilizers, or other materials well known to those skilled in the art and optionally other therapeutic agents. The formulations (also referred to herein as compositions or pharmaceutical compositions) may conveniently be presented in unit dosage form and may be prepared by any known methods. All formulations and pharmaceutical compositions, as well as any methods of using such pharmaceutical compositions, disclosed herein are contemplated and considered to be within the scope of the disclosure provided herein.

Administration of the agents and pharmaceutical compositions described herein can be effected by any method that enables delivery of the agents to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the agent, and other suitable methods. For example, agents and pharmaceutical compositions described herein can be administered locally to the area in need of treatment. Administration is achieved by, by way of non-limiting example, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue. Those of ordinary skill in the art are familiar with formulation and administration techniques that can be employed with the agents and methods of the invention, e.g., as discussed in Goodman and Gilman, The Pharmacological Basis of Therapeutics (current edition); Pergamon and Remington's, Pharmaceutical Sciences, Mack Publishing Co., Easton, Pa. (current edition).

The pharmaceutical compositions included herein are those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrasternal), intraperitoneal, transmucosal, transdermal, rectal and topical (including dermal, buccal, sublingual, intranasal, intraocular, and vaginal) administration. The most suitable mode of administration is determined based on the condition of the patient and the specific disorder targeted. In certain embodiments, the pharmaceutical compositions described herein are conveniently formulated in unit dosage form and may be prepared by any of the methods well known in the art of pharmacy. All methods include the step of bringing into association an agent (“active ingredient”) or combination of agents (“active ingredients”) with the carrier which constitutes one or more accessory ingredients. In general, formulations are prepared by uniformly and intimately bringing into association the active ingredient with liquid carriers or finely divided solid carriers or both and then, if necessary, shaping the product into the desired formulation.

In certain embodiments, formulations suitable for oral administration are presented as discrete units such as capsules, cachets or tablets each containing a predetermined amount of the active ingredient or ingredients; as a powder or granules; as a solution or a suspension in an aqueous liquid or a non-aqueous liquid; or as an oil-in-water liquid emulsion or a water-in-oil liquid emulsion. In other embodiments, the active ingredient or ingredients are presented as a bolus, electuary or paste.

In some embodiments, formulations suitable for oral administration include tablets, push-fit capsules made of gelatin, as well as soft, sealed capsules made of gelatin and a plasticizer, such as glycerol or sorbitol. In certain embodiments, tablets are made by compression or molding, optionally with one or more accessory ingredients. In some embodiments, compressed tablets are prepared by compressing in a suitable machine the active ingredient in a free-flowing form such as a powder or granules, optionally mixed with binders (e.g., povidone, gelatin, hydroxypropylmethyl cellulose), inert diluents, preservative, disintegrant (e.g., sodium starch glycolate, cross-linked povidone, cross-linked sodium carboxymethyl cellulose) or lubricating, surface active or dispersing agents. In other embodiments, molded tablets are made by molding in a suitable machine a mixture of the powdered active ingredient or ingredients moistened with an inert liquid diluent. The tablets are optionally coated or scored. In certain embodiments, tablets are formulated so as to provide slow or controlled release of the active ingredient therein. Tablets are optionally provided with an enteric coating, to provide release in parts of the gut other than the stomach. All formulations for oral administration should be in dosages suitable for such administration. In other embodiments, the push-fit capsules contain the active ingredient or ingredients in admixture with filler such as lactose, binders such as starches, and/or lubricants such as talc or magnesium stearate and, optionally, stabilizers. In certain embodiments, soft capsules contain the active ingredient or ingredients dissolved or suspended in suitable liquids, such as fatty oils, liquid paraffin, or liquid polyethylene glycols. In addition, stabilizers are optionally added. Dragee cores are provided with suitable coatings. For this purpose, concentrated sugar solutions may be used, which optionally contains gum arabic, talc, polyvinyl pyrrolidone, carbopol gel, polyethylene glycol, and/or titanium dioxide, lacquer solutions, and suitable organic solvents or solvent mixtures. Dyestuffs, pigments or other color agents are optionally to the tablets or Dragee coatings for identification (e.g., as a pharmaceutical composition comprising the first agent, the second agent or a combination of first and second agents) or to characterize different doses.

In other embodiments, pharmaceutical compositions are formulated for parenteral administration by injection, e.g., by bolus injection or continuous infusion. In various embodiments, formulations for injection are presented in unit dosage form, e.g., in ampoules or in multi-dose containers, with an optional preservative. In certain embodiments, formulations take forms including, by way of non-limiting example, suspensions, solutions or emulsions in oily or aqueous vehicles, and optionally contain formulatory agents such as suspending, stabilizing and/or dispersing agents. In some embodiments, the formulations are presented in unit-dose or multi-dose containers, for example, sealed ampoules and vials. In some embodiments, the formulations are stored in powder form or in a freeze-dried (lyophilized) condition requiring only the addition of the sterile liquid carrier, for example, saline or sterile pyrogen-free water, immediately prior to use. Extemporaneous injection solutions and suspensions are prepared, by way of non-limiting example, from sterile powders, granules and tablets of the kind previously described.

Formulations for parenteral administration include aqueous and non-aqueous (oily) sterile injection solutions of the active agents which may contain antioxidants, buffers, biocide, bacteriostats and solutes which render the formulation isotonic with the blood of the intended recipient; and aqueous and non-aqueous sterile suspensions which optionally include suspending agents and thickening agents. Examples of suitable isotonic vehicles for use in such formulations include Sodium Chloride Injection, Ringer's Solution, or Lactated Ringer's Injection. Suitable lipophilic solvents or vehicles include fatty oils such as sesame oil, or synthetic fatty acid esters, such as ethyl oleate or triglycerides, or liposomes or other microparticulate systems may be used to target the compound to blood components or one or more organs. The concentration of the active ingredient or ingredients in the solution varies depending on intended usage.

As such, the invention further provides pharmaceutical compositions and methods of making said pharmaceutical composition. In some embodiments, the pharmaceutical compositions comprise an effective amount of the first and second agents. In other embodiments, a first pharmaceutical composition comprises the first agent and a second pharmaceutical composition comprises the second agent. The pharmaceutical composition may comprise admixing at least one active ingredient with one or more carriers, excipients, buffers, adjuvants, stabilizers, or other materials well known to those skilled in the art and optionally other therapeutic agents. The formulations may conveniently be presented in unit dosage form and may be prepared by any known methods.

Non-limiting examples of excipients that are used in conjunction with the present invention include water, saline, dextrose, glycerol or ethanol. The injectable compositions optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Example of pharmaceutically acceptable carriers that are optionally used include, but are not limited to aqueous vehicles, no aqueous vehicles, antimicrobial agents, isotonic agents, buffers, antioxidants, local anesthetics, suspending and dispersing agents, emulsifying agents, sequestering or chelating agents and other pharmaceutically acceptable substances.

In some embodiments, pharmaceutical compositions are formulated as a depot preparation. In certain embodiments, such long acting formulations are administered by implantation (for example subcutaneously or intramuscularly) or by intramuscular injection. Thus, in various examples, the agents or combinations described herein are formulated with suitable polymeric or hydrophobic materials (for example as an emulsion in an acceptable oil) or ion exchange resins, or as sparingly soluble derivatives, for example, as a sparingly soluble salt.

In other embodiments, wherein the pharmaceutical compositions described herein are formulated for buccal or sublingual administration, the pharmaceutical compositions described herein takes the form of tablets, lozenges, pastilles, or gels formulated in conventional manner. Such compositions optionally flavored agents such as sucrose and acacia or tragacanth.

In still other embodiments of the present invention, pharmaceutical compositions are formulated in rectal compositions such as suppositories or retention enemas, e.g., containing conventional suppository bases such as cocoa butter, polyethylene glycol, or other glycerides.

In yet other embodiments, pharmaceutical compositions are administered topically. Topical administration includes non-systemic administration. In certain embodiments, the active ingredient or ingredients are applied externally to the epidermis or the buccal cavity and the instillation of such a agent into the ear, eye and nose, such that the agent does not significantly enter the blood stream. In alternative embodiments, the pharmaceutical compositions described herein are delivered systemically, which includes oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. In alternative embodiments, a formulation comprises a patch or a dressing such as a bandage or adhesive plaster impregnated with the active ingredient or ingredients and optionally one or more excipients or diluents.

Formulations suitable for topical administration in the mouth include lozenges comprising the active ingredient or ingredients in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient or ingredients are dissolved or suspended in a suitable carrier, including an aqueous solvent.

Formulations for administration by inhalation are conveniently delivered from an insufflators, nebulizer pressurized packs or other convenient means of delivering an aerosol spray. Pressurized packs optionally comprise a suitable propellant such as dichlorodifluoromethane, trichlorofluoromethane, dichlorotetrafluoroethane, carbon dioxide or other suitable gas. In certain aspects of pressurized aerosols, the dosage unit is determined by providing a valve to deliver a metered amount. In alternative embodiments, for administration by inhalation or insufflation, formulations take the form of a dry powder composition, for example a powder mix of the agent and a suitable powder base such as lactose or starch. In certain embodiments, the powder composition is presented in unit dosage form, in for example, capsules, cartridges, gelatin or blister packs from which the powder may be administered with the aid of an inhalator or insufflator.

It should be understood that in addition to the ingredients particularly mentioned above, the agents and compositions described herein may include other agents or components conventional in the art having regard to the type of formulation in question, for example, those suitable for oral administration may include flavoring agents.

In certain embodiments, the agents or pharmaceutical compositions described herein are delivered in a vesicle, e.g., a liposome. In various embodiments, the agents and pharmaceutical compositions described herein are delivered in a controlled release system. In one embodiment, a pump is used. In additional embodiments, a controlled release system is placed in proximity of the therapeutic target. In certain aspects of the present invention, the pharmaceutical compositions described are formulated into a formulation suitable for oral use, for example, as tablets, troches, lozenges, aqueous or oily suspensions, dispersible powders or granules, emulsions, hard or soft capsules, or syrups or elixirs. Pharmaceutical compositions intended for oral use are prepared according to any method known to the art for the manufacture of pharmaceutical compositions. In order to provide pharmaceutically elegant and palatable preparations pharmaceutical compositions described herein optionally contain one or more agents selected from the group consisting of sweetening agents, flavoring agents, coloring agents and preserving agents. Tablets contain the active ingredient or ingredients in admixture with one or more non-toxic pharmaceutically acceptable excipient which is suitable for the manufacture of tablets. Excipients include, by way of non-limiting example, inert diluents (e.g., calcium carbonate, sodium carbonate, lactose, calcium phosphate or sodium phosphate), granulating and disintegrating agents (e.g., microcrystalline cellulose, sodium croscarmellose, corn starch, or alginic acid), binding agents (e.g., starch, gelatin, polyvinyl-pyrrolidone or acacia), and lubricating agents (e.g., magnesium stearate, stearic acid or talc). The tablets are optionally coated or uncoated. Coating of a tablet is accomplished by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. In alternative embodiments, formulations for oral use are in the form of hard gelatin capsules wherein the active ingredient or ingredients are mixed with an inert solid diluent. Suitable inert solid diluents include, by way of non-limiting example, calcium carbonate, calcium phosphate or kaolin. In further embodiments, formulations for oral use are in the form of soft gelatin capsules wherein the active ingredient or ingredients are mixed with water soluble carrier. Water soluble carriers include, by way of non-limiting example, polyethyleneglycol or an oil medium (e.g., peanut oil, liquid paraffin, or olive oil).

Aqueous suspensions contain the active material in admixture with one or more excipient suitable for the manufacture of aqueous suspensions. Suitable excipients include, by way of non-limiting example, suspending agents (e.g., sodium carboxymethylcellulose, methylcellulose, hydroxypropylmethyl-cellulose, sodium alginate, polyvinyl-pyrrolidone, gum tragacanth and gum acacia), dispersing or wetting agents (e.g., a naturally-occurring phosphatide such as lecithin, condensation products of an alkylene oxide with fatty acids such as polyoxyethylene stearate, condensation products of ethylene oxide with long chain aliphatic alcohols such as heptadecaethylene-oxycetanol, condensation products of ethylene oxide with partial esters derived from fatty acids and a hexitol such as polyoxyethylene sorbitol monooleate, or condensation products of ethylene oxide with partial esters derived from fatty acids and hexitol anhydrides, such as polyethylene sorbitan monooleate). The aqueous suspensions optionally contain one or more preservatives (e.g., ethyl, or n-propyl p-hydroxybenzoate), one or more coloring agents, one or more flavoring agents, and one or more sweetening agents (e.g., sucrose, saccharin or aspartame).

In various embodiments, oily suspensions are formulated by suspending the active ingredient in, by way of non-limiting example, a vegetable oil (e.g., arachis oil, olive oil, sesame oil or coconut oil), or in mineral oil (e.g., liquid paraffin). The oily suspensions optionally contain a thickening agent (e.g., beeswax, hard paraffin or cetyl alcohol). Sweetening agents such as those set forth above, and flavoring agents are optionally added to provide a palatable oral preparation. Preservatives and/or anti-oxidants (e.g., butylated hydroxyanisol or alpha-tocopherol) are optionally added as well.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional optional excipients include, by way of non-limiting example, sweetening, flavoring, coloring agents and anti-oxidants. Anti-oxidants include ascorbic acid.

In certain embodiments, pharmaceutical compositions are formulated as oil-in-water emulsions. The oily phase is selected from, by way of non-limiting example, vegetable oil (e.g., olive oil or arachis oil), a mineral oil (e.g., liquid paraffin) or mixtures thereof. Suitable emulsifying agents include naturally-occurring phosphatides (e.g., soy bean lecithin), esters or partial esters derived from fatty acids and hexitol anhydrides (e.g., sorbitan monooleate), and condensation products of the said partial esters with ethylene oxide (e.g., polyoxyethylene sorbitan monooleate). The emulsions optionally contain sweetening agents, flavoring agents, preservatives and antioxidants.

Syrups and elixirs are optionally formulated with sweetening agents (e.g., glycerol, propylene glycol, sorbitol or sucrose). Such formulations also optionally contain one or more demulcent, one or more preservative, one or more flavoring agent, one or more coloring agent and/or one or more antioxidant.

In another embodiment, pharmaceutical compositions are in the form of a sterile injectable aqueous solution. Acceptable vehicles and solvents that are employed are, by way of non-limiting example, water, Ringer's solution and isotonic sodium chloride solution. In some embodiments, the sterile injectable preparation is a sterile injectable oil-in-water microemulsion where the active ingredient is dissolved in the oily phase. In an example, the active ingredient is dissolved in a mixture of soybean oil and lecithin. The oil solution is then introduced into a water and glycerol mixture and processed to form a microemulsion. The injectable solutions or microemulsions may be introduced into a patient's blood-stream by local bolus injection. Alternatively, it may be advantageous to administer the solution or microemulsion in such a way as to maintain a constant circulating concentration of the instant active ingredient or ingredients. In order to maintain such a constant concentration, a continuous intravenous delivery device is utilized in some embodiments. An example of such a device is the Deltec CADD-PLUS™ model 5400 intravenous pump. In other embodiments of the present invention, the pharmaceutical compositions is in the form of a sterile injectable aqueous or oleaginous suspension for intramuscular and subcutaneous administration. This suspension is formulated according to the known art using suitable dispersing, wetting agents and/or suspending agents, all of which are discussed herein. In still other embodiments, the sterile injectable preparation is a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In other embodiments of the present invention, pharmaceutical compositions are administered in the form of suppositories for rectal administration of the drug. In some embodiments, these pharmaceutical compositions are prepared by mixing the active ingredient or ingredients with a suitable non-irritating excipient which is solid at ordinary temperatures but liquid at the rectal temperature and will therefore melt in the rectum to release the drug. Such materials include, by way of non-limiting example, cocoa butter, glycerinated gelatin, hydrogenated vegetable oils, mixtures of polyethylene glycols of various molecular weights and fatty acid esters of polyethylene glycol.

In still other embodiments, the pharmaceutical compositions described herein are formulated for topical use, creams, ointments, jellies, solutions or suspensions, etc., containing an agent or a pharmaceutical composition described herein is used. As used herein, topical application(s) include mouth washes and gargles.

In yet other embodiments, pharmaceutical compositions are administered in intranasal form via topical use of suitable intranasal vehicles and delivery devices, or via transdermal routes, using those forms of transdermal skin patches well known to those of ordinary skill in the art. Tansdermal delivery system, include continuous administration of the active ingredient or ingredients.

In certain embodiments, the pharmaceutical compositions described herein are formulated as a form suitable for oral administration, as a tablet, as a capsule, as a cachet, as a pill, as a lozenge, as a powder or as a granule. In some embodiments of the present invention, the pharmaceutical compositions are formulated as sustained release formulations, solutions, liquids, suspensions, for parenteral injection as a sterile solution, suspension or emulsion, for topical administration as an ointment, cream, lotions, sprays, foams, gel or paste, or for rectal or vaginal administration as a suppository or pessary. In certain embodiments, the pharmaceutical compositions are formulated in unit dosage forms suitable for single administration of precise dosages. In certain aspects, the pharmaceutical composition includes a conventional pharmaceutical carrier or excipient and an agent as described herein as an active ingredient. In addition, other medicinal or pharmaceutical agents, carriers, adjuvants, etc., are included.

Exemplary parenteral administration forms include solutions or suspensions of active agents in sterile aqueous solutions, for example, aqueous propylene glycol or dextrose solutions. Such dosage forms are optionally buffered.

Suitable pharmaceutical carriers include inert diluents or fillers, water and various organic solvents. The pharmaceutical compositions optionally contain additional ingredients such as flavorings, binders, excipients and the like. For example, in a specific embodiment, tablets containing various excipients, such as citric acid are employed together with various disintegrants. Disintegrants include, by way of non-limiting example, starch or other cellulosic material, alginic acid and certain complex silicates and with binding agents such as sucrose, gelatin and acacia. Additionally, lubricating agents such as magnesium stearate, sodium lauryl sulfate and talc are optionally used. Other reagents such as an inhibitor, surfactant or solubilizer, plasticizer, stabilizer, viscosity increasing agent, or film forming agent are also optionally added. In certain embodiments, solid compositions of a similar type are employed in soft and hard filled gelatin capsules. In certain embodiments, the pharmaceutical compositions and/or formulations described herein include lactose or milk sugar and high molecular weight polyethylene glycols. When aqueous suspensions or elixirs are desired for oral administration the active ingredient or ingredients are optionally combined with various sweetening or flavoring agents, coloring agents or dyes and, emulsifying agents or suspending agents, together with diluents such as water, ethanol, propylene glycol, glycerin, or combinations thereof.

### Kits

The pharmaceutical compositions and methods described herein may be included in kits for the treatment of HDAC mediated disorders, such as cancer. These kits may include a Class I selective HDAC inhibitor and second HDAC inhibitor or pharmaceutical compositions thereof in a container and, optionally, instructions teaching the use of the kit according to the various methods and approaches described herein. The kits may also include additional therapeutic agents, as described below.

Such kits optionally include information, such as scientific literature references, package insert materials, clinical trial results, and/or summaries of these and the like, which indicate or establish the activities and/or advantages of the composition, and/or which describe dosing, administration, side effects, drug interactions, disease state for which the composition is to be administered, or other information useful to the health care provider. In some embodiments, the kit includes information and/or instructions regarding dosage and administration regimens regarding the sequential administration of the first agent (i.e. the Class I selective HDAC inhibitor) and the second agent (i.e. the second HDAC inhibitor) as described in the Method of Treatment section above. Such information may be based on the results of various studies, for example, studies using experimental animals involving in vivo models and studies based on human clinical trials. In various embodiments, the kits described herein can be provided, marketed and/or promoted to health providers, including physicians, nurses, pharmacists, formulary officials, and the like. Kits are, in some embodiments, marketed directly to the consumer. In certain embodiments, the packaging material further comprises a container for housing the composition and optionally a label affixed to the container. The kit optionally comprises additional components, such as, by way of non-limiting example, syringes for administration of the composition.

In some embodiments, a kit for HDAC mediated diseases is provided. Thus, in some embodiments, the kit is for treating cancer. The kit includes a first pharmaceutical composition comprising a first amount of a Class I selective HDAC inhibitor and a second pharmaceutical composition comprising a second amount of a second HDAC inhibitor. The elements of pharmaceutical compositions are described above in the Pharmaceutical Compositions and Formulations section and are equally applicable to the pharmaceutical compositions in the kits provided herein. The histone deacetylase mediated disorders, Class I selective HDAC inhibitors, second HDAC inhibitors, the first amount, the second amount, and cancers described above in the Method of Treatment section are equally applicable to the pharmaceutical compositions, formulations and kits described herein.

The second pharmaceutical composition may an oral dosage form comprising a prolonged release portion. The prolonged release portion typically includes the second HDAC inhibitor. Prolonged release portions are described above in the Pharmaceutical Compositions and Formulations section and are equally applicable to the pharmaceutical compositions in the kits provided herein.

As described above, in some embodiments, the first amount and the second amount are together an effective amount to provide a synergistic therapeutic anti-cancer effect. The discussion above related to synergistic therapeutic anti-cancer effects are equally applicable to the pharmaceutical compositions and formulations described herein.

As also described above, in some embodiments, the second amount of the second HDAC inhibitor is less than a therapeutic amount of the second HDAC inhibitor when the second HDAC inhibitor is administered without the Class I selective HDAC inhibitor. And as also described above, the first amount of the Class I selective HDAC inhibitor may be less than a therapeutic amount of the Class I selective HDAC when the Class I selective HDAC inhibitor is administered without the second HDAC inhibitor.

In some embodiments, the Class I selective HDAC inhibitor is entinostat, sodium phenyl butyrate, MGCD-0103, FK228, spiruchostatin A, SK7041, SK7068 or a 6 amino nicotinamide. The Class I selective HDAC inhibitor may also simply be entinostat. The second HDAC inhibitor may be vorinostat, pyroxamide, CBHA, trichostatin A, trichostatin C, salicylihydroxamic acid, azelaic bihydroxamic acid, azelaic-1-hydroxamate-9-analide, 6-(3-chlorophenylureido) carpoic hydroxamic acid (3Cl UCHA), oxamflatin, A 161906, scriptaid, PXD-101, LAQ-824, CHAP, MW2796, LBH589 or MW2996. In some embodiments, the second HDAC inhibitor is vorinostat or trichostatin C. In other embodiments, the second HDAC inhibitor is vorinostat.

In some embodiments, the first pharmaceutical composition and the second pharmaceutical composition are independently selected from an injectable dosage form and a oral dosage form. Injectable dosage forms and a oral dosage forms are described above in the Pharmaceutical Compositions and Formulations section and are equally applicable to the pharmaceutical compositions in the kits provided herein. In some embodiments, the first pharmaceutical composition and the second pharmaceutical composition are injectable dosage forms.

In a some embodiments, the kit comprises (1) a first pharmaceutical composition that contains a therapeutically effective amount of a first agent and a therapeutically effective amount of the second agent, and (2) a second pharmaceutical composition that contains a therapeutically effective amount of the second agent and does not contain a therapeutically effective amount of the first agent. In another specific embodiment, the kit comprises (1) a first pharmaceutical composition that contains a therapeutically effective amount of a first agent and does not contain a therapeutically effective amount of the second agent, and (2) a second pharmaceutical composition that contains a therapeutically effective amount of the second agent and a therapeutically effective amount of the first agent. In various embodiments, the kit contains an additional agent.

In some embodiments, the kit comprises a first pharmaceutical composition that is visibly different from a second pharmaceutical composition. The visible differences may be for example shape, size, color, state (e.g., liquid/solid), physical markings (e.g., letters, numbers) and the like. In certain embodiments, the kit comprises a first pharmaceutical composition that is a first color and a second pharmaceutical composition that is a second color. In embodiments wherein the first and second colors are different, the different colors of the first and second pharmaceutical compositions is used, e.g., to distinguish between the first and second pharmaceutical compositions. In further embodiments, a third pharmaceutical composition containing an additional agent is a third color.

In some embodiments, wherein the packaging material further comprises a container for housing the pharmaceutical composition, the kit comprises a first pharmaceutical composition that is in a different physical location within the kit from a second pharmaceutical composition. In some embodiments, the different physical locations containing the first and second pharmaceutical compositions comprise separately sealed individual compartments. In certain embodiments, the kit comprises a first pharmaceutical composition that is in a first separately sealed individual compartment and a second pharmaceutical composition that is in a second separately sealed individual compartment. In embodiments wherein the first and second compartments are separate, the different locations of the first and second pharmaceutical compositions are used, e.g., to distinguish between the first and second pharmaceutical compositions. In further embodiments, a third pharmaceutical composition is in a third physical location within the kit.

### Assays

In another aspect, the present invention provides assays to determine whether a combination of a Class I selective HDAC inhibitor and a second HDAC inhibitor have a synergistic therapeutic anti-cancer effect. As defined above, a “synergistic therapeutic anti-cancer effect” means that a given combination of at least 2 compounds exhibits synergy when tested in an anti-cancer assay.

In some embodiments, synergy is assessed using the median-effect principle (Chou, et al., Adv Enzyme Regul 22:27-55 (1984)). This method is based on Michaelis-Menton kinetics and reduces combination effects to a numeric indicator, the combination index (C.I.). Where the combination index is less than 1, synergism is indicated. Where the combination index is equal to 1, summation is indicated. Where the combination index is greater than 1, antagonism is indicated. It may be possible to boserve mixed effects over a range of C.I. values. Therefore, in some embodiments, only combinations that are consistent over at least the majority of the drug concentration range are classified as synergistic, additive, or antagonistic. Examples of assays useful in assessing synergistic therapeutic anti-cancer effects are provided below in this section as well as in the Examples section (e.g. Examples 6, 7, and 8).

In an exemplary embodiment, the combination index of a Class I selective HDAC inhibitor and a second HDAC inhibitor is less than 1.0. In another exemplary embodiment, the combination index of a Class I selective HDAC inhibitor and a second HDAC inhibitor is at least less than 0.9. In another exemplary embodiment, the combination index of a Class I selective HDAC inhibitor and a second HDAC inhibitor is at least less than 0.8. In another exemplary embodiment, the combination index of a Class I selective HDAC inhibitor and a second HDAC inhibitor is at least less than 0.7. In another exemplary embodiment, the combination index of a Class I selective HDAC inhibitor and a second HDAC inhibitor is at least less than 0.6.

Various anti-cancer assays are well-known in the art. For example, a number of biological assays are available to evaluate and to optimize the choice of specific combinations of compounds for optimal antitumor activity. These assays can be roughly split into two groups those involving in vitro exposure of agents to tumor cells and in vivo antitumor assays in rodent models and rarely, in larger animals. Both in vitro assay using tumor cells and in vivo assays in animal models are discussed below, and are equally applicable to determining whether an thiol-binding mitochondrial oxidant, a nucleic acid binding agent, or an antimetabolite base analog, exhibit antineoplastic properties.

Cytotoxic assays in vitro for a combination of a Class I selective HDAC inhibitor and a second HDAC inhibitor generally involve the use of established tumor cell lines both of animal and, especially of human origin. These cell lines can be obtained from commercial sources such as the American Type Tissue Culture Laboratory in Bethesda, Md. and from tumor banks at research institutions. Exposures to combinations of the present invention may be carried out under simulated physiological conditions of temperature, oxygen and nutrient availability in the laboratory. The endpoints for these in vitro assays can involve: 1) colony formation; 2) a simple quantitation of cell division over time; 3) the uptake of so called “vital” dyes which are excluded from cells with an intact cytoplasmic membrane (or reduction of dyes); 4) the incorporation of radiolabeled nutrients into a proliferating (viable) cell. Colony forming assays have been used both with established cell lines, as well as fresh tumor biopsies surgically removed from patients with cancer. In this type of assay, cells are typically grown in petri dishes on soft agar, and the number of colonies or groups of cells are counted either visually, or with an automated image analysis system. A comparison is then made to the untreated control cells allowed to develop colonies under identical conditions. Because colony formation is one of the hallmarks of the cancer phenotype, only malignant cells will form colonies without adherence to a solid matrix. This can therefore be used as a screening procedure for the combinations provided herein, and there are a number of publications which show that results obtained in colony forming assays correlates with clinical trial findings with the same drugs.

The enumeration of the total number of cells is a simplistic approach to in vitro testing with either cell lines or fresh tumor biopsies. In this assay, clumps of cells are typically disaggregated into single units which can then be counted either manually on a microscopic grid or using an automated flow system such as either flow cytometry or a Coulter® counter. Control (untreated) cell growth rates are then compared to the treated (with a combination of antineoplastic thiol-binding mitochondrial oxidant and a second antineoplastic agent) cell growth rates. Vital dye staining is another one of the older hallmarks of antitumor assays. In this type of approach cells, either untreated or treated with a cancer drug, are subsequently exposed to a dye such as methylene blue, which is normally excluded from intact (viable) cells. The number of cells taking up the dye (dead or dying) are the numerator with a denominator being the number of cells which exclude the dye. These are laborious assays which are not currently used extensively due to the time and the relatively non-specific nature of the endpoint.

In addition to vital dye staining, viability can be assessed using the incorporation of radiolabeled nutrients and/or nucleotides. In tumor cell assays, a typical experiment involves the incorporation of either (³H) tritium or ¹⁴C-labeled nucleotides such as thymidine. Control (untreated) cells are shown to take up a substantial amount of this normal DNA building block per unit time, and the rate of incorporation is compared to that in the drug treated cells. This is a rapid and easily quantitatable assay that has the additional advantage of working well for cells that may not form large (countable) colonies. Drawbacks include the use of radioisotopes which present handling and disposal concerns.

There are large banks of human and rodent tumor cell lines that are available for these types of assays. The current test system used by the National Cancer Institute uses a bank of numerous established sensitive and multidrug-resistant human cells lines of a variety of cell subtypes. This typically involves many established and well-characterized human tumor cells of a particular subtype for testing combinations. Using a graphic analysis systems, the overall sensitivity in terms of dye uptake or dye reduction (either sulforhodamine B or MTT tetrazolium dye) may be utilized. The specific goal of this approach is to identify combinations that are uniquely active in a single histologic subtype of human cancer.

Generally, once a combination has demonstrated synergistic activity in vitro at inhibiting tumor cell growth, such as colony formation or dye uptake (or reduction), antitumor efficacy experiments may be performed in vivo. Rodent systems are almost exclusively used for initial assays of antitumor activity since tumor growth rates and survival endpoints are well-defined, and since these animals generally reflect the same types of toxicity and drug metabolism patterns as in humans. For this work, syngeneic (same gene line) tumors are typically harvested from donor animals, disaggregated, counted and then injected back into syngeneic (same strain) host mice. Anticancer combinations are typically then injected at some later time point(s), either by intraperitoneal, intravenous or administered by the oral routes, and tumor growth rates and/or survival are determined, compared to untreated controls or controls having only a Class I selective HDAC inhibitor or a second HDAC inhibitor. In these assays, growth rates are typically measured for tumors injected growing in the front flank of the animal, wherein perpendicular diameters of tumor width are translated into an estimate of total tumor mass or volume. The time to reach a predetermined mass is then compared to the time required for equal tumor growth in the untreated control animals. In some embodiments, significant findings generally involve a >25% increase in the time to reach the predetermined mass in the treated animals compared to the controls. In other embodiments, significant findings involve a >42% increase in the time to reach the predetermined mass in the treated animals compared to the controls. The significant findings are termed tumor growth inhibition. For non-localized tumors such as leukemia, survival can be used as an endpoint and a comparison is made between the treated animals and the untreated or solvent treated controls. In general, a significant increase in life span for a positive combination may be >20-42% longer life span due to the treatment. Early deaths, those occurring before any of the untreated controls, generally indicate toxicity for a new compound.

For all these assays, the anticancer combinations may be tested at doses very near the lethal dose and 10% (LD₁₀) and/or at the determined maximally-tolerated dose, that dose which produces significant toxicity, but no lethality in the same strain of animals and using the same route of administration and schedule of dosing. Similar studies can also be performed in rat tumor models although, because of the larger weight and difficulty handling these animals they are less preferred than the murine models.

Human tumors have been successfully transplanted in a variety of immunologically deficient mouse models. A “nude” mouse may be employed for in vivo assays of human tumor growth. In nude mice, which are typically hairless and lack a functional thymus gland, human tumors (millions of cells) are typically injected in the flank and tumor growth occurs slowly thereafter. This visible development of a palpable tumor mass is called a “take”. Anticancer combinations are then injected by some route (IV, IM, IP, SQ, PO) distal to the tumor implant site, and growth rates are calculated by perpendicular measures of the widest tumor widths as described earlier. A number of human tumors are known to successfully “take” in the nude mouse model, even though these animals are more susceptible to intercurrent infections due to the underlying immunologic deficiency. An alternative mouse model for this work involves mice with a severe combined immunodeficiency disease (SCID) wherein there is a defect in maturation of lymphocytes. Because of this, SCID mice do not produce functional B- and T-lymphocytes. However, these animals do have normal cytotoxic T-killer cell activity. Nonetheless, SCID mice will “take” a large number of human tumors. Animals with the SCID phenotype are screened for “leakiness” by measuring serum immunoglobulin production which should be minimal to undetectable if the SCID phenotype is maintained. Tumor measurements and drug dosing are generally performed as above. The use of SCID mice has in many cases displaced the nude mouse since SCID mice seem to have a greater ability to take a larger number of human tumors and are more robust in terms of lack of sensitivity to intercurrent infections. Positive combinations in the SCID mouse model are those that inhibit tumor growth rate by >20-42% compared to the untreated control.

All of these test systems are generally combined in a serial order, moving from in vitro to in vivo, to characterize the antitumor activity of an anticancer combination. In general, one wishes to find out what tumor types are particularly sensitive to a combination and conversely what tumor types are intrinsically resistant to a combination in vitro. Using this information, experiments are then planned in rodent models to evaluate whether or not the combinations that have shown activity in vitro will be tolerated and active in animals. The initial experiments in animals generally involve toxicity testing to determine a tolerable dose schedule and then using that dose schedule, to evaluate antitumor efficacy as described above. Active combinations from these two types of assays may then be tested in human tumors growing in SCID or nude mice and if activity is confirmed, these combinations then become candidates for potential clinical drug development.

### Additional Therapeutic Agents

In certain embodiments, the methods, compositions and kits disclosed herein provide an additional therapeutic agent. For example, the methods of the present invention may additionally include the administration of an additional therapeutic agent. A pharmaceutical composition may additionally include a third amount (e.g. a therapeutically effective amount) of an additional therapeutic agent. And a kit may include a third pharmaceutical composition comprising a third amount of an additional agent.

Thus, in some embodiments of the present invention, a method of treating cancer with a Class I selective HDAC inhibitor and a second HDAC inhibitor comprises administering an additional chemotherapeutic agent (e.g. cancer therapy) to a patient. In certain embodiments, the additional cancer therapy is, by way of non-limiting example, surgery, radiation therapy or at least one chemotherapeutic agent. The at least one chemotherapeutic agent includes, by way of non-limiting example, one or more of adriamycin, gemcitabine, mitomycin C, cisplatin, carboplatin, oxaliplatin, fluorouracil, leucovorin, cytarabine, etoposide, capecitabine, temozolomide, doxorubicin, daunomycin, daunorubicin, paclitaxel, docetaxel, cyclophosphamide, ifosfamide, methotrexate, bevacizumab, and trastuzumab. Other chemotherapeutic agents are set forth below and are equally applicable to the compositions and kits set forth herein.

In some embodiments, the additional therapeutic agent (also referred to herein as an additional agent) is used to treat inflammation and/or pain. In various embodiments, the additional therapeutic agent is, by way of non-limiting example, a corticosteroid, a non-steroidal anti-inflammatory agent, a muscle relaxant or combinations thereof. In other embodiments, the additional therapeutic agent is, by way of non-limiting example, an anesthetic, an expectorant, an antidepressant, an anticonvulsant, an antihypertensive, an opioid, a cannabinoid, capsaicin, or combinations thereof. In some embodiments, the additional agent is not an HDAC inhibitor.

In other embodiments of the present invention, an additional therapeutic agent is selected from betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocalne, EMLA Cream (Eutectic Mixture of Local Anesthetics (lidocaine 2.5% and prilocalne 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, inhibitors of inducible nitric oxide synthase; capsaicin or combinations thereof is administered.

In certain embodiments, an additional therapeutic agent is selected from beta-blockers, carbonic anhydrase inhibitors, α- and β-adrenergic antagonists including a1-adrenergic antagonists, α2 agonists, miotics, prostaglandin analogs, corticosteroids, immunosuppressant agents, timolol, betaxolol, levobetaxolol, carteolol, levobunolol, propranolol, brinzolamide, dorzolamide, nipradilol, iopidine, brimonidine, pilocarpine, epinephrine, latanoprost, travoprost, bimatoprost, unoprostone, dexamethasone, prednisone, methylprednisolone, azathioprine, cyclosporine, immunoglobulins, and combinations thereof is administered.

In still other embodiments, the present invention provides a method for treating autoimmune disorders. In certain embodiments, an agent selected from corticosteroids, immunosuppressants, prostaglandin analogs and antimetabolites, dexamethasone, prednisone, methylprednisolone, azathioprine, cyclosporine, immunoglobulins, latanoprost, travoprost, bimatoprost, unoprostone, infliximab, rutuximab, methotrexate and combinations thereof is administered.

In certain embodiments, an additional agent selected from insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, non-glitazone type PPARS agonist, tformin, Glipizide, glyburide, Amaryl, meglitinides, nateglinide, repaglinide, PT-112, SB-517955, SB4195052, SB-216763, NN-57-05441, NN-57-05445, GW-0791, AGN-.sup.194.sup.204, T-1095, BAY R3401, acarbose Exendin-4, DPP728, LAF237, vildagliptin, MK-0431, saxagliptin, GSK23A, pioglitazone, rosiglitazone, (R)-1-{4-[5-methyl-2-(4-trifluoromethyl-phenyl)-oxazol-4-ylmethoxy]-benze-nesulfonyl}2,3-dihydro-1H-indole-2-carboxylic acid, GI-262570 and combinations thereof is administered.

In further embodiments, an additional agent selected from corticosteroids, non-steroidal anti-inflammatories, muscle relaxants, anesthetics, expectorants, antidepressants, anticonvulsants, antihypertensives, opioids, topical cannabinoids, and capsaicin is administered.

In some embodiments, the additional agent is a proteasome inhibitor. Proteasome inhibitors include, by way of non-limiting example, bortezomib (Velcade, PS-341), PR-171, NPI-0052 (salinosporamide A), MG-132, omuralide, lactacystin and NEOSH101. In certain embodiments, the proteasome inhibitor is, by way of non-limiting example, bortezomib (Velcade, PS-341), PR-171 (carfilzomib), (benzyloxycarbonyl)-Leu-Leu-phenylalaninal, 2,3,5a,6-tetrahydro-6-hydroxy-3-(hydroxymthyl)-2-methyl-10H-3a,10a-epidithio-pyrazinol[1,2a]indole-1,4-dione, 4-hydroxy-3-nitrophenylacetyl-Leu-Leu-Leu-vinyl sulphone, sapojargon, Ac-hFLFL-epoxide, aclacinomycin A, aclarubicin, ACM, AdaK(Bio)Ahx3L3VS, AdaLys(Bio)Ahx3L3VS, Adamantane-acetyl-(6-aminohexanoyl)-3-(leucunyl)-3-vinyl-(methyl)-sulphone, ALLM, ALLN, Calpain Inhibitor I, Calpain Inhibitor II, Carbobenzoxy-L-leucyl-L-leucyl-L-leucinal, Carbobenzoxy-L-leucyl-L-leucyl-L-norvalinal, gliotoxin, isovalery-L-tyrosyl-L-valyl-DL-tyrosinal, clasto-lactacystin-β-lactone, Z-LL-Nva-CHO, Ubiquitin Aldehyde, YU101, MP-LLL-VS, LDN-57444, Z-GPFL-CHO, Z-LLL-CHO, lovastatin, α-methyl-clasto-lactacystin-P-lactone, mevinolin, MK-803, NIP-L3VS, NP-LLL-VS, NPI-0052 (salinosporamide A), MLN519 (PS-519), NLVS (trileucine vinyl-sulfone), ritonavir, Ro106-9920, Z-LLF-CHO, Z-LL-B(OH)2, RRRPRPPYLPR, Tyropeptin A, ZL3VS, PR-11, PR-39, 0106-9920, Proteasome Inhibitor I, Proteasome Inhibitor II, Proteasome Inhibitor III, Proteasome Inhibitor IV, AdaAhx3L3VS, efrapeptin, MG-132, MG-262, MG-115, α-methylomuralide, MG-101, epoxomicin, omuralide, lactacystin and/or NEOSH101. In certain embodiments, the proteasome inhibitor is bortezomib.

In other embodiment, the additional agent is a kinase inhibitor with anti-EGFR activity. In some embodiments, the kinase inhibitor with anti-EGFR activity is a dual kinase inhibitor. Tyrosine kinase inhibitors are also contemplated herein. Examples of kinase inhibitors with anti-EGFR activity include, by way of non-limiting example, gefitinib (IRESSA®, ZD1839), erlotinib (TARCEVA®, OSI-774, CP-358), or PKI-166; EGFR-specific and irreversible inhibitors, such as EKI-569; a PAN-HER (human EGF receptor family) reversible inhibitor, such as GW2016 (targets both EGFR and Her2/neu); and a PAN-HER irreversible inhibitor, such as CI-1033 (4-anilinoquinazoline).

Furthermore, in certain embodiments, the tyrosine kinase inhibitor and EGFR antagonist is, by way of non-limiting example, small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037 (each of which are incorporated by reference herein in their entireties). In additional embodiments, the second agent is an EGFR antagonist selected from, by way of non-limiting example, PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZM 105180 ((6-amino-4-(3-methylphenyl-amino)-quinazoline, Zeneca); BIBX-1382 (N-8-(3-chloro-4-fluoro-phenyl)-N-2-(1-methyl-piperidin-4-yl)-pyrimido[5,-4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)-; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); Imatinib; STI-571; LFM-A13; PD153035; Piceatannol; PP1, Lapatinib (Tykerb®, GW572016, GlaxoSmithKline); AEE788; SU4132; SU6656; Semazanib; SU6668, ZD6126 AG1478 (Sugen); Vandetanib (ZACTIMA™; ZD6474) and AG1571 (SU 5271; Sugen).

In some embodiments, the additional agent is a chemotherapeutic agent. Non-limiting examples of chemotherapeutic agents are anticancer agents, alkylating agents, cytotoxic agents, antimetabolic agents, hormonal agents, plant-derived agents, and biologic agents. In certain embodiments, the second agent is a chemotherapeutic agent selected from, by way of non-limiting example, adriamycin, gemcitabine, mitomycin, oxaliplatin, fluorouracil, leucovorin, cytarabine, etoposide, capecitabine, temozolomide, doxorubicin, paclitaxel, docetaxel, bevacizumab or trastuzumab.

Anti-tumor substances are selected from, by way of non-limiting example, mitotic inhibitors (e.g., vinblastine), alkylating agents (e.g., cis-platin, carboplatin and cyclophosphamide), anti-metabolites (5-fluorouracil, cytosine arabinside and hydroxyurea), one of the anti-metabolites disclosed in European Patent Application No. 239362 (e.g., N-(5-[N-(3,4-dihydro-2-methyl-4-oxoquinazolin-6-ylmethyl)-N-methylamino]-2-thenoyl)-L-glutamic acid), growth factor inhibitors, cell cycle inhibitors, intercalating antibiotics (e.g, adriamycin and bleomycin), enzymes (e.g., interferon), anti-hormones (e.g., anti-estrogens such as Nolvadex™ (tamoxifen) or anti-androgens such as Casodex™ (4′-cyano-3-(4-fluorophenylsulphonyl)-2-hydroxy-2-methyl-3′-(trifluoromethyl) propionanilide)). As with any treatment regiment described herein, these chemotherapeutic agents are administered, in various embodiments, simultaneous, sequential or separate from the first agent.

Alkylating agents include, by way of non-limiting example, bischloroethylamines (nitrogen mustards, e.g., chlorambucil, cyclophosphamide, ifosfamide, mechlorethamine, melphalan, uracil mustard), aziridines (e.g., thiotepa), alkyl alkone sulfonates (e.g., busulfan), nitrosoureas (e.g., carmustine, lomustine, streptozocin), nonclassic alkylating agents (altretamine, dacarbazine, and procarbazine), platinum compounds (carboplastin and cisplatin).

Cytotoxic agents include, by way of non-limiting example, anthracyclines (e.g., doxorubicin, daunorubicin, epirubicin, idarubicin and anthracenedione), mitomycin C, bleomycin, dactinomycin, plicatomycin.

Antimetabolic agents are a group of drugs that interfere with metabolic processes vital to the physiology and proliferation of cancer cells. Antimetabolic agents include, by way of non-limiting example, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine.

In other embodiments, the additional agent is a hormonal agent. Harmonal agents are a group of drug that regulate the growth and development of their target organs. Hormonal agents include sex steroids and their derivatives and analogs thereof, such as estrogens, androgens, and progestins. Hormonal agents include, by way of non-limiting example, synthetic estrogens (e.g., diethylstibestrol), antiestrogens (e.g., tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone.

In other embodiments, the additional agent is plant-derived agent including, by way of non-limiting example, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis.

In other embodiments, the additional agent is a biologic agent. As used herein, the phrase “biologic agents” refers to a group of biomolecules that elicit cancer/tumor regression when used alone or in combination with chemotherapy and/or radiotherapy. Biologic agents include, by way of non-limiting example, immuno-modulating proteins such as cytokines, monoclonal antibodies against tumor antigens, tumor suppressor genes, and cancer vaccines.

Furthermore, in various embodiments of the present invention, a chemotherapeutic agent selected from, by way of non-limiting example, aromatase inhibitors, antiestrogen, anti-androgen, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, anti-angiogenic agents, Akt inhibitors, IGF-I inhibitors (including monoclonal antibodies and small molecule inhibitors), FGF3 modulators, mTOR inhibitors, Smac mimetics, other HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, caminomycin, idarubicin, epirubicin, esorubicin, mitoxantrone, bleomycins A, A₂, and B, camptothecin, Irinotecan®, Topotecan®, 9-aminocamptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin®, Rituxan®, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva®, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel is administered.

In further embodiments, the additional agent is interleukin 2 (IL-2), interleukin 4 (IL-4), or interleukin 12 (IL-12) is administered.

In other embodiments, the additional agent is an interferon. Interferons include more than 23 related subtypes with overlapping activities, all of the IFN subtypes within the scope of the present invention. IFN has demonstrated activity against many solid and hematologic malignancies, the later appearing to be particularly sensitive.

Other cytokine additional agent include cytokines that exert profound effects on hematopoiesis and immune functions. Examples of such cytokines include, by way of non-limiting example, erythropoietin, granulocyte-CSF (filgrastin), and granulocyte, macrophage-CSF (sargramostim).

Other immuno-modulating additional agents include, by way of non-limiting example, bacillus Calmette-Guerin, levamisole, and octreotide, a long-acting octapeptide that mimics the effects of the naturally occurring hormone somatostatin.

In other embodiments, the additional agent is a monoclonal antibody. Monoclonal antibodies against tumor antigens are antibodies elicited against antigens expressed by tumors, preferably tumor-specific antigens. Monoclonal antibodies of the present invention include, by way of non-limiting example, HERCEPTIN® and RITUXAN®

In other embodiments, the additional agent is a tumor suppressor gene. As used herein, tumor suppressor genes are genes that function to inhibit the cell growth and division cycles, thus preventing the development of neoplasia. Tumor suppressor genes include, by way of non-limiting example, DPC-4, NF-1, NF-2, RB, p53, WT1, BRCA1 and BRCA2.

In other embodiments, the additional agent is a cancer vaccine. Cancer vaccines are a group of agents that induce the body's specific immune response to tumors. Most of cancer vaccines under research and development and clinical trials are tumor-associated antigens (TAAs). TAA are structures (i.e., proteins, enzymes or carbohydrates) which are present on tumor cells and relatively absent or diminished on normal cells. By virtue of being fairly unique to the tumor cell, TAAs provide targets for the immune system to recognize and cause their destruction. TAAs include, by way of non-limiting example, gangliosides (GM2), prostate specific antigen (PSA), alpha-fetoprotein (AFP), carcinoembryonic antigen (CEA) (produced by colon cancers and other adenocarcinomas, e.g., breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

As set forth above, the first and second agents described may be administered with one or more additional therapeutic agent. In these embodiments, either or both of the first and second agents described herein can be in a fixed combination with an additional therapeutic agent or a non-fixed combination with an additional therapeutic agent. In other words, in some embodiments, an additional therapeutic agent is combined with the first agent. In other embodiments, an additional therapeutic agent is combined with the second agent. In still other embodiments, the additional therapeutic agent is administered separately from either the first or second agents. In yet other embodiments, the therapeutic agent is formulated with both the first and second agents in a single formulation. In other embodiments, the first agent is formulated into a first pharmaceutical composition that further comprises an additional therapeutic agent and the second agent is formulated into a second pharmaceutical composition that also contains an additional therapeutic agent. In still other embodiments, the first agent is formulated into a first pharmaceutical composition that does not comprise an additional therapeutic agent and the second agent is formulated into a second pharmaceutical composition that does contain an additional therapeutic agent. In yet other embodiments, the first agent is formulated into a first pharmaceutical composition that further comprises an additional therapeutic agent and the second agent is formulated into a second pharmaceutical composition that does not contain an additional therapeutic agent. In certain embodiments, the different pharmaceutical compositions are distinguished by color (e.g., by using different coloring agents in each of the pharmaceutical compositions utilized). Provided below are various embodiments of additional therapeutic agents that are combined with the first and second agents described hereinabove.

As used herein, any reference to an additional therapeutic agent refers to one or more additional therapeutic agents. As such, in one embodiment, provided herein is a method of treating a histone deacetylase mediated disorder with a first agent, a second agent, and an additional therapeutic agent. In another embodiment, provided herein is a method of treating a histone deacetylase mediated disorder with a first agent, a second agent, a first additional therapeutic agent, and a second additional therapeutic agent.

In one embodiment of the present invention, the additional therapeutic agent is an anti-hypertensive agent. In other embodiments of the present invention, the additional therapeutic agent is an agent that enhances the efficacy of either or both of the first and second agents.

Therapies include, but are not limited to, administration of other therapeutic agents, radiation therapy or both. In the instances where the first and/or second agents described herein are administered with other therapeutic agents, the agents described herein need not be administered in the same pharmaceutical composition as any additional therapeutic agents. Furthermore, in various embodiments, the first agent, second agent and any additional therapeutic agent are administered by different routes. In other embodiments, one or more of the first agent, second agent and any additional therapeutic agent is administered by the same route. In still other embodiments, each of the first agent, second agent and any additional therapeutic agent are administered by the same route. In one example, one or more of the agents is administered orally, while one or more of the other agents are administered intravenously. In further embodiments, the dosage, modes of administration and times of administration of one or more of the agents is modified after administration is begun.

In certain embodiments, the first agent, second agent, and where applicable additional therapeutic agents are administered concurrently (e.g., simultaneously, essentially simultaneously or within the same treatment protocol). In other embodiments, the first agent, second agent, and where applicable additional therapeutic agent are administered sequentially. In still other embodiments, certain agents are administered concurrently while others are administered sequentially. The manner in which the agents are delivered depends on the nature of the disease, the condition of the patient, and/or the choice of additional therapeutic agent and/or therapy (e.g., radiation) to be administered. Furthermore, it is to be understood that these administration methods include the administration of one or all of the agents in a pharmaceutical composition as described herein.

In combinational applications and uses, the first agent, second agent and the additional therapeutic agent need not be administered simultaneously or essentially simultaneously. Indeed, in some embodiments, the initial order of administration of the agents or pharmaceutical compositions thereof is not important. Thus, in certain embodiments, the first and second agent or pharmaceutical compositions thereof are administered prior to the administration of the additional therapeutic agent. In another embodiment, the additional therapeutic agent is administered prior to the first and second agents. In still another embodiment, the first agent is administered first, the additional therapeutic agent is administered second, and the second agent is administered third. In various embodiments, a treatment protocol repeats the sequence of steps described or combines them. In certain embodiments, the treatment protocol is repeated until treatment is complete. In further embodiments, as treatment proceeds a treatment protocol is modified according to the individual patient's needs. Indications of the patient's needs include, but are not limited to, relief of disease-related symptoms, inhibition of tumor growth, actual shrinkage of the tumor, or inhibition of metastasis. Tumor size is measured by standard methods, including radiological studies (e.g., CAT or MRI scan).

In certain embodiments, the present invention provides a method of treating cancer by first administering a chemotherapeutic agent (i.e., the additional therapeutic agent) or combination of chemotherapeutic agents, followed by the administration of the first and second agents. In such embodiments, the first and second agents are administered according to any regimen, including simultaneously or sequentially. In some embodiments, the chemotherapeutic agent is administered as a mono-therapy or combination therapy over the course of a period of time (including, e.g., a week, two weeks, a month, 2 months, 3 months, 6 months), after which the steps of administering the first and second agents commence. In certain embodiments, the chemotherapeutic agent(s) is/are administered for a time sufficient to result in a reduction in tumor size or volume of burden, after which the steps of administering the first and second agents commence. In some embodiments, the first and second agents are administered following the chemotherapeutic agent in order to maintain the size or control of a tumor that was reduced due to administration of the chemotherapeutic agent. In traditional chemotherapy treatment plans, the later therapy with the agents herein would be described as “maintenance therapy” and may continue for a defined period of time or indefinitely.

Specific, non-limiting examples of additional therapeutic agents are found in the pharmacotherapeutic classifications listed below. These lists are illustrative only and are not to be construed as limiting. Moreover, as with the first and second agents, the additional therapeutic agent is administered in any acceptable manner including, by way of non-limiting example, oral, intravenous, intraocular, subcutaneous, dermal, and inhaled topical. As with the first and second agents, the additional therapeutic agent need not be administered in a manner identical to either or both of the first and second agents.

In some embodiments, additional therapeutic agents include chemotherapeutic agents. As with any treatment regiment described herein, these chemotherapeutic agents are administered, in various embodiments, simultaneous, sequential or separate from either or both of the first and second agents.

In certain embodiments, an adjuvant is used in the combination to augment the immune response to TAAs. Examples of adjuvants include, by way of non-limiting example, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan.

In some embodiments of the invention, the treatments and uses described herein carry with them side effects that include, for example, nausea, vomiting, immunosuppression and susceptibility to infections, anemia and pain. Therefore, in certain embodiments, the additional therapeutic agent is any agent that abrogates treats, reduces the incidence of or prevents such side effects. In some embodiments, additional therapeutic agents include, by way of non-limiting example, anti-emetic agents, immuno-restorative agents, antibiotic agents, anemia treatment agents, and analgesic agents for treatment of pain and inflammation.

As used herein anti-emetic agents are defined as drugs effective for treatment of nausea and emesis (vomiting). Anti-emetic agents include, by way of non-limiting example, 5-HT³ seratonin receptor antagonists. 5-HT3 antagonists include, by way of non-limiting example, dolasetron (Anzemet®), granisetron (Kytril®), ondansetron (Zofran®), palonosetron and tropisetron. Other anti-emetic agents include, by way of non-limiting example, dopamine receptor antagonists (e.g., chlorpromazine, domperidone, droperidol, haloperidol, metaclopramide, promethazine, and prochlorperazine), antihistamines (e.g., cyclizine, diphenhydramine, dimenhydrinate, meclizine, promethazine, and hydroxyzine), lorazepram, scopolamine, dexamethasone, Emetrol®, propofol, and trimethobenzamide.

As used herein, immuno-restorative agents are defined as drugs that counter the immuno-suppressive effects of cancer therapies. Immuno-restorative agents include, by way of non-limiting example, synthetic analogs of the hormone, granulocyte colony stimulating factor (G-CSF), filgrastim (Neupogen®), PEG-filgrastim (Neulasta®) and lenograstim.

As used herein, antibiotic agents include drugs that have anti-bacterial, anti-fungal, and anti-parasite properties. Antibiotics include, by way of non-limiting example, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin.

In further embodiments, the additional therapeutic agent is selected from, by way of non-limiting example, corticosteroids, non-steroidal anti-inflammatories, muscle relaxants, anesthetics, expectorants, antidepressants, anticonvulsants, antihypertensives, opioids, topical cannabinoids, and capsaicin.

It is noted that any reference to the administration of a first agent, a second agent, an additional therapeutic agent, or any combination thereof includes the administration of a pharmaceutical composition comprising the agent or agents disclosed as being administered.

### EXAMPLES

Below are provided non-limiting examples of the present invention.

### Example 1

### Evaluation of Synergistic Effect in Colorectal Carcinoma

The following is an example the evaluation of the synergistic effect of a first HDAC inhibitor in combination with a second HDAC inhibitor in colorectal carcinoma (CRC) in vivo. The activity of a first HDAC inhibitor as single agent and in combination with a second HDAC inhibitor is evaluated in nude mice bearing CRC cell lines. Mice bearing CRC tumors are randomly assigned to treatment groups, and the effect of MS-275, SAHA, and an MS-275/SAHA combination on tumor growth is evaluated. Nude mice are implanted with CRC cell-lines. Implantation of a tumor is achieved through established tumor transplantation techniques (e.g., injection or surgical orthotopic implantation). Upon establishment of the CRC tumor, as determined by tumor volume measurement, the effect of MS-275, SAHA, and a combination of MS-275 and SAHA is evaluated for inhibition of tumor growth. Each agent (MS-275, SAHA, or MS-275/SAHA combination) is administered to different groups of mice in different dosages. Each agent is administered as follows: MS-275—2 doses, SAHA—2 doses, MS-275/SAHA combination—4 doses. Biopsies and measurements of the tumors are taken at 4 time points corresponding to 0, 48, 72, and 96 hours post-treatment. Tumor volumes are measured for each time point to determine efficacy of the agents.

### Example 2

### Evaluation of Synergistic Effect in Breast Cancer

The following is an example the evaluation of the synergistic effect of a first HDAC inhibitors in combination with a second HDAC inhibitor in breast cancer in vivo. The activity of a first HDAC inhibitor as single agent and in combination with a second HDAC inhibitor is evaluated in nude mice bearing breast cancer cell lines. Mice bearing breast cancer tumors are randomly assigned to treatment groups, and the effect of MS-275, SAHA, and an MS-275/SAHA combination on tumor growth is evaluated. Nude mice are implanted with breast cancer cell lines. Implantation of a tumor is achieved through established tumor transplantation techniques (e.g., injection or surgical orthotopic implantation). Upon establishment of the breast cancer tumor, as determined by tumor volume measurement, the effect of MS-275, SAHA, and a combination of MS-275 and SAHA is evaluated for inhibition of tumor growth. Each agent (MS-275, SAHA, or MS-275/SAHA combination) is administered to different groups of mice in different dosages. Each agent is administered as follows: MS-275—2 doses, SAHA—2 doses, MS-275/SAHA combination—4 doses. Biopsies and measurements of the tumors are taken at 4 time points corresponding to 0, 48, 72, and 96 hours post-treatment. Tumor volumes are measured for each time point to determine efficacy of the agents.

### Example 3

### Treatment with MS-275 and SAHA

Human Clinical Trial of the Safety and/or Efficacy of MS-275/SAHA combination therapy

Objective: To compare the safety and pharmacokinetics of administered MS-275 and SAHA.
Study Design: This will be a Phase I, single-center, open-label, randomized dose escalation study followed by a Phase II study in cancer patients with disease that can be biopsied (i.e., breast cancer, non-small cell lung cancer, prostate cancer, pancreatic cancer, colorectal cancer, head cancer and neck cancer). Patients should not have had exposure to MS-275 or SAHA prior to the study entry. Patients must not have received treatment for their cancer within 2 weeks of beginning the trial. Treatments include the use of chemotherapy, hematopoietic growth factors, and biologic therapy such as monoclonal antibodies. The exception is the use of hydroxyurea for patients with WBC>30×103/μL. This duration of time appears adequate for wash out due to the relatively short-acting nature of most anti-leukemia agents. Patients must have recovered from all toxicities (to grade 0 or 1) associated with previous treatment. All subjects are evaluated for safety and all blood collections for pharmacokinetic analysis are collected as scheduled. All studies are performed with institutional ethics committee approval and patient consent.

Phase I: Patients receive oral SAHA daily for 5 consecutive days or 7 days a week and oral MS-275 on days 1, 8, and 15 or 1 and 15. Doses of either SAHA or SNDX-275 may be held or modified for toxicity based on assessments as outlined below. Treatment repeats every 28 days in the absence of unacceptable toxicity. Cohorts of 3-6 patients receive escalating doses of SAHA and MS-275 until the maximum tolerated dose (MTD) for the combination of SAHA and MS-275 is determined. Test dose ranges are initially determined via the established individual dose ranges for MS-275 and SAHA. A standard dosage for SAHA is 200-400 mg per day (or 175-230 mg/m² per dose), given daily for 5 consecutive or 7 days per week. An established dosage for MS-275 includes 2-4 mg/m² per dose. Additional dosages, both decreasing and increasing in amount as well a frequency, are determined based on the standard dose for both MS-275 and SAHA. The MTD is defined as the dose preceding that at which 2 of 3 or 2 of 6 patients experience dose-limiting toxicity. Dose limiting toxicities are determined according to the definitions and standards set by the National Cancer Institute (NCI) Common Terminology for Adverse Events (CTCAE) Version 3.0 (Aug. 9, 2006).

Phase II: Patients receive SAHA as in phase I at the MTD determined in phase I and MS-275 as in phase I. Treatment repeats every 6 weeks for 2-6 courses in the absence of disease progression or unacceptable toxicity. After completion of 2 courses of study therapy, patients who achieve a complete or partial response may receive an additional 4 courses. Patients who maintain stable disease for more than 2 months after completion of 6 courses of study therapy may receive an additional 6 courses at the time of disease progression, provided they meet original eligibility criteria.

Blood Sampling: Serial blood is drawn by direct vein puncture before and after administration of MS-275 or SAHA. Venous blood samples (5 mL) for determination of serum concentrations are obtained at about 10 minutes prior to dosing and at approximately the following times after dosing: days 1, 2, 3, 4, 5, 6, 7, and 14. Each serum sample is divided into two aliquots. All serum samples are stored at −20° C. Serum samples are shipped on dry ice.

Pharmacokinetics: Patients undergo plasma/serum sample collection for pharmacokinetic evaluation before beginning treatment and at days 1, 2, 3, 4, 5, 6, 7, and 14. Pharmacokinetic parameters are calculated by model independent methods on a Digital Equipment Corporation VAX 8600 computer system using the latest version of the BIOAVL software. The following pharmacokinetics parameters are determined: peak serum concentration (Cₘₐₓ); time to peak serum concentration (tₘₐₓ); area under the concentration-time curve (AUC) from time zero to the last blood sampling time (AUC₀₋₇₂) calculated with the use of the linear trapezoidal rule; and terminal elimination half-life (t_{1/2}), computed from the elimination rate constant. The elimination rate constant is estimated by linear regression of consecutive data points in the terminal linear region of the log-linear concentration-time plot. The mean, standard deviation (SD), and coefficient of variation (CV) of the pharmacokinetic parameters are calculated for each treatment. The ratio of the parameter means (preserved formulation/non-preserved formulation) is calculated.

Patient Response to combination therapy: Patient response is assessed via imaging with X-ray, CT scans, and MRI, and imaging is performed prior to beginning the study and at the end of the first cycle, with additional imaging performed every four weeks or at the end of subsequent cycles. Imaging modalities are chosen based upon the cancer type and feasibility/availability, and the same imaging modality is utilized for similar cancer types as well as throughout each patient's study course. Response rates are determined using the RECIST criteria. (Therasse et al., J. Natl. Cancer Inst., 92(3):205-16 (2000); http://ctep.cancer.gov/forms/TherasseRECISTJNCI.pdf). Patients also undergo cancer/tumor biopsy to assess changes in progenitor cancer cell phenotype and clonogenic growth by flow cytometry, Western blotting, and IHC, and for changes in cytogenetics by FISH. After completion of study treatment, patients are followed periodically for 4 weeks.

In conclusion, administration of a combination of MS-275 and SAHA will be safe and well tolerated by cancer patients. The combination of MS-275 and SAHA provides large clinical utility to cancer patients.

### Example 4

### Parenteral Composition

An i.v. solution is prepared in a sterile isotonic solution of water for injection and sodium chloride (˜300 mOsm) at pH 11.2 with a buffer capacity of 0.006 mol/l/pH unit. The protocol for preparation of 100 ml of a 5 mg/ml, a first and/or second agent for i.v. infusion, is as follows: add 25 ml of NaOH (0.25 N) to 0.5 g of a first and/or second agent and stir until dissolved without heating. Add 25 ml of water for injection and 0.55 g of NaCl and stir until dissolved. Add 0.1N HCl slowly until the pH of the solution is 11.2. The volume is adjusted to 100 ml. The pH is checked and maintained between 11.0 and 11.2. The solution is subsequently sterilized by filtration through a cellulose acetate (0.22 μm) filter before administration.

### Example 5

### Oral Composition

A pharmaceutical composition for oral delivery is prepared by mixing 100 mg of a first and/or second agent with 750 mg of a starch. The mixture is incorporated into an oral dosage unit, such as a hard gelatin capsule or coated tablet, which is suitable for oral administration.

Many modifications, equivalents, and variations of the present invention are possible in light of the above teachings, therefore, it is to be understood that within the scope of the appended claims, the invention may be practiced other than as specifically described.

### Example 6

### Determination of Anti-Tumor Activity

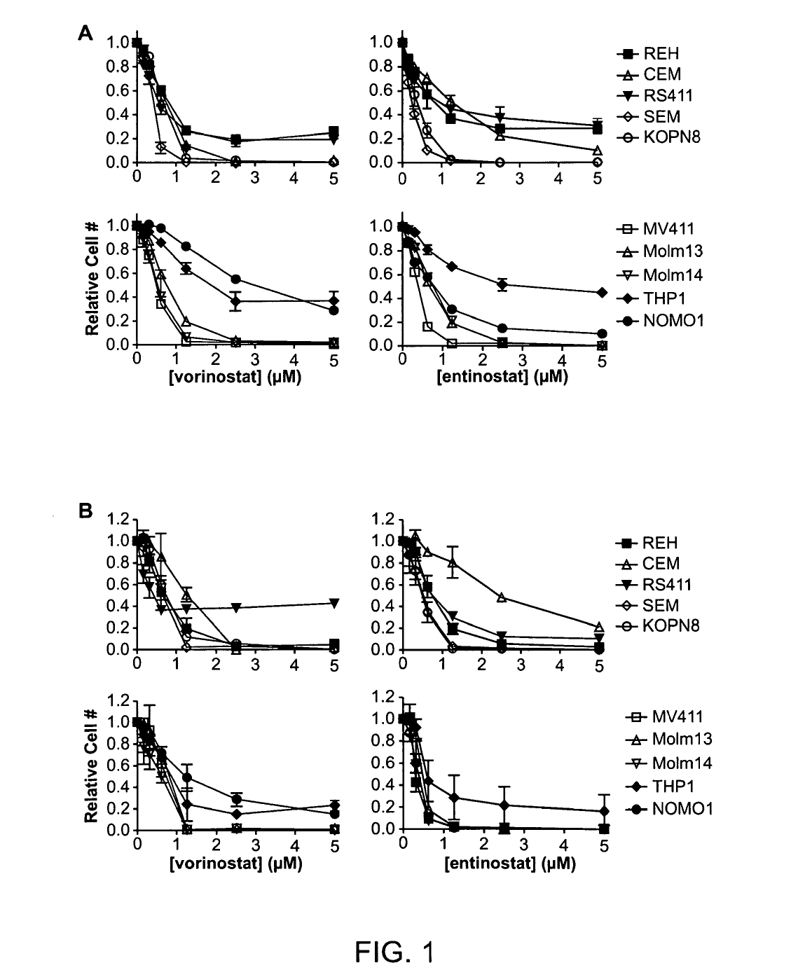

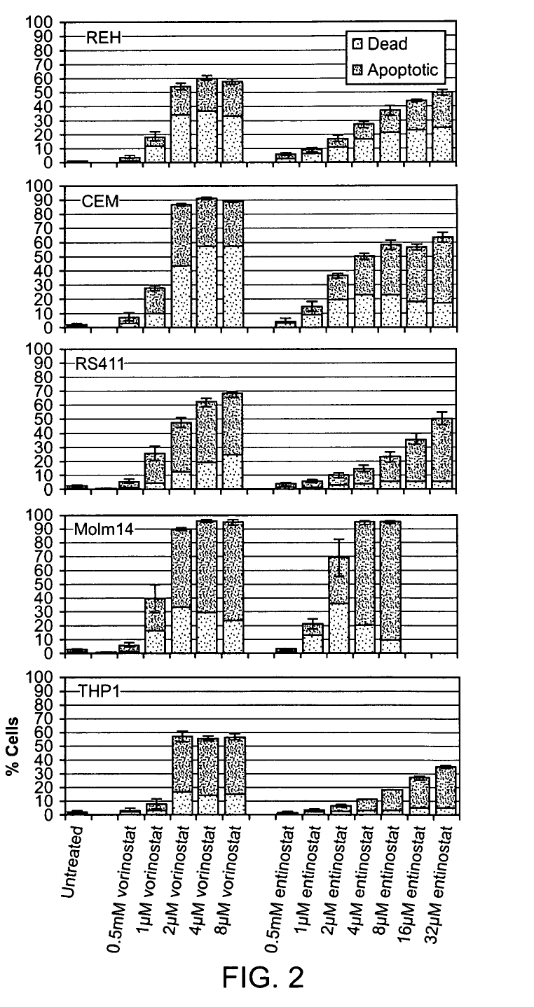

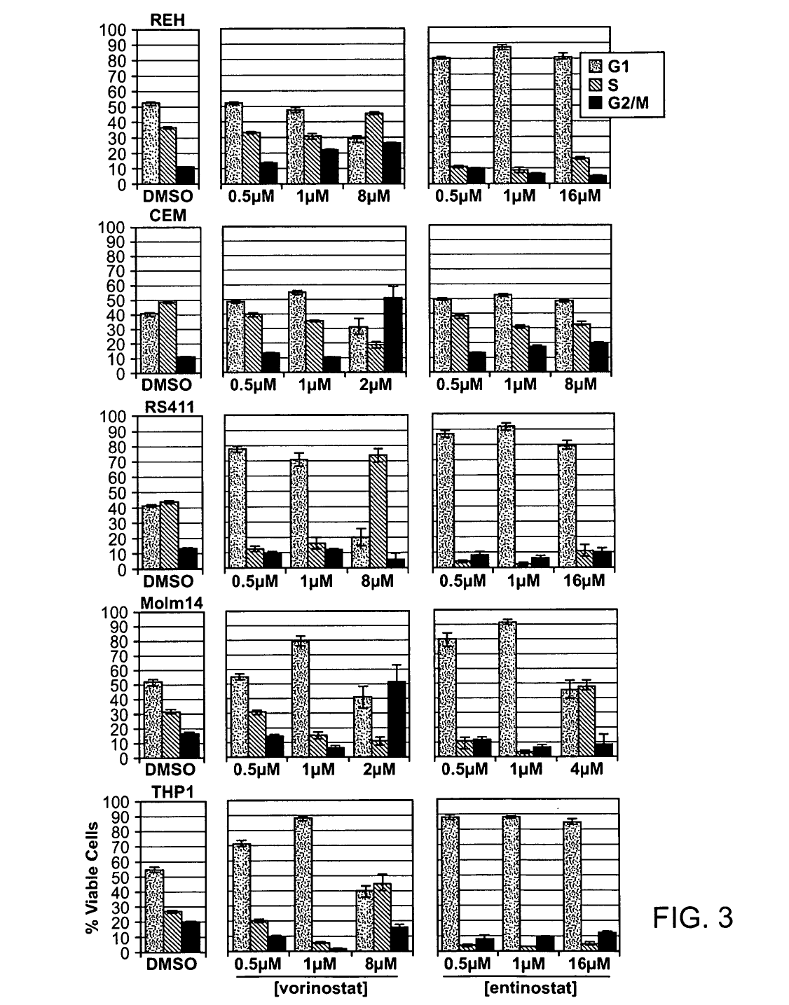

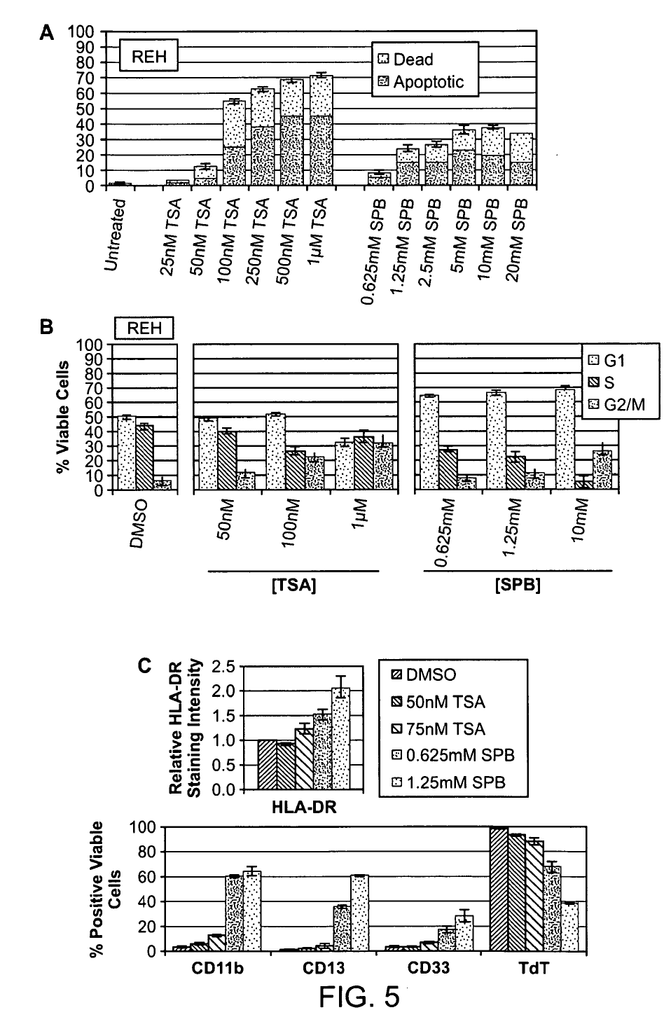

Acute lymphocytic leukemia (ALL) cell lines (REH, CEM, RS411, SEM, and/or KOPN8) and acute myeloid leukemia (AML) cell lines (MV411, Molm13, Molm14, THP1, and/or NOMO1) were treated with vorinostat (SAHA), entinostat (SNDX-275), or vehicle only for 48 hours (FIGS. 1-3 and Table 1). Alternatively, an ALL cell line (REH) was treated with Trichostatin A (TSA), sodium phenyl butyrate (SPB), or vehicle only for 48 hours. MTT or BrdU were added for 4 hours. The relative number of viable cells, as indicated by MTT reduction, was determined (FIG. 1A and Table 1). The relative number of proliferating cells, as indicated by BrdU-incorporation, was measured using an ELISA assay (FIG. 1B). IC50 values were calculated using non-linear regression (Table 1). The percentages of apoptotic and dead cells were determined by flow cytometric analysis of cells stained with YO-PRO®-1 and propidium iodide (FIGS. 2 and 5A). DNA content was determined by flow cytometric analysis of propidium iodide stained cells. The fractions of cells with G1, S, and G2/M phase DNA contents were determined (FIGS. 3 and 5B).

TABLE 1
Anti-tumor activity of histone deacetylase inhibitors
against established acute leukemia cell lines.

| | | IC₅₀ +/− SEM (nM) | |
|---|---|---|---|
| Cell Line | Genetic Alteration | vorinostat | entinostat |

T Cell Lymphoblastic Leukemia

| | | | |
|---|---|---|---|
| Jurkat | | 810 +/− 85 | 1274 +/− 156 |
| Molt-3 | | 574 +/− 91 | 866 +/− 258 |
| Molt-4 | | 548 +/− 65 | 721 +/− 40 |
| HSB-2 | LCK-TCRβ | 941 +/− 91 | 1286 +/− 205 |
| CEM | | 710 +/− 51 | 1285 +/− 105 |

B Cell Lymphoblastic Leukemia

| | | | |
|---|---|---|---|
| RCH-AcV | E2A-PBX1 | 529 +/− 55 | 723 +/− 103 |
| REH | TEL-AML1 | 704 +/− 53 | 1500 +/− 415 |
| UOCB1 | E2A-HLF | 477 +/− 49 | 686 +/− 138 |
| Nalm-6 | PDGFRβ-TEL | 643 +/− 61 | 801 +/− 52 |
| HAL-01 | TCF3/E2A-HLF | 486 +/− 63 | 499 +/− 90 |

Biphenotypic Leukemia with Lymphoblastic Features

| | | | |
|---|---|---|---|
| RS4; 11 | MLL-AF4 | 466 +/− 44 | 2420 +/− 338 |
| SEM | MLL-AF4 | 410 +/− 36 | 250 +/− 27 |
| KOPN-8 | MLL-ENL | 646 +/− 25 | 396 +/− 73 |

Myeloid Leukemia

| | | | |
|---|---|---|---|
| HEL | | 625 +/− 44 | 957 +/− 120 |
| KG1 | | 477 +/− 35 | 1541 +/− 465 |
| U937 | | 723 +/− 63 | 1003 +/− 180 |
| EOL-1 | FIP1L1-PDGFRα | 377 +/− 63 | 120 +/− 10 |
| HL-60 | | 1214 +/− 49 | 690 +/− 160 |

Biphenotypic Leukemia with Myeloid Features

| | | | |
|---|---|---|---|
| MV4-11 | MLL-AF4 | 468 +/− 32 | 362 +/− 15 |
| Molm-13 | MLL-AF9 | 725 +/− 33 | 758 +/− 37 |
| Molm-14 | MLL-AF9 | 538 +/− 43 | 839 +/− 48 |
| THP-1 | MLL-AF9 | 2525 +/− 601 | 4453 +/− 479 |
| NOMO-1 | MLL-AF9 | 1637 +/− 208 | 587 +/− 40 |

Cultures of the indicated cell lines were exposed to various concentrations of vorinostat or entinostat for 48 hours and cell numbers relative to cultures treated with vehicle only were determined by MTT assay. Dose-response curves were generated and IC₅₀ values were determined by nonlinear regression. Mean IC₅₀ values and standard errors were derived from 4-11 independent experiments.

### Example 7

### Determination of Differentiation Marker Expression

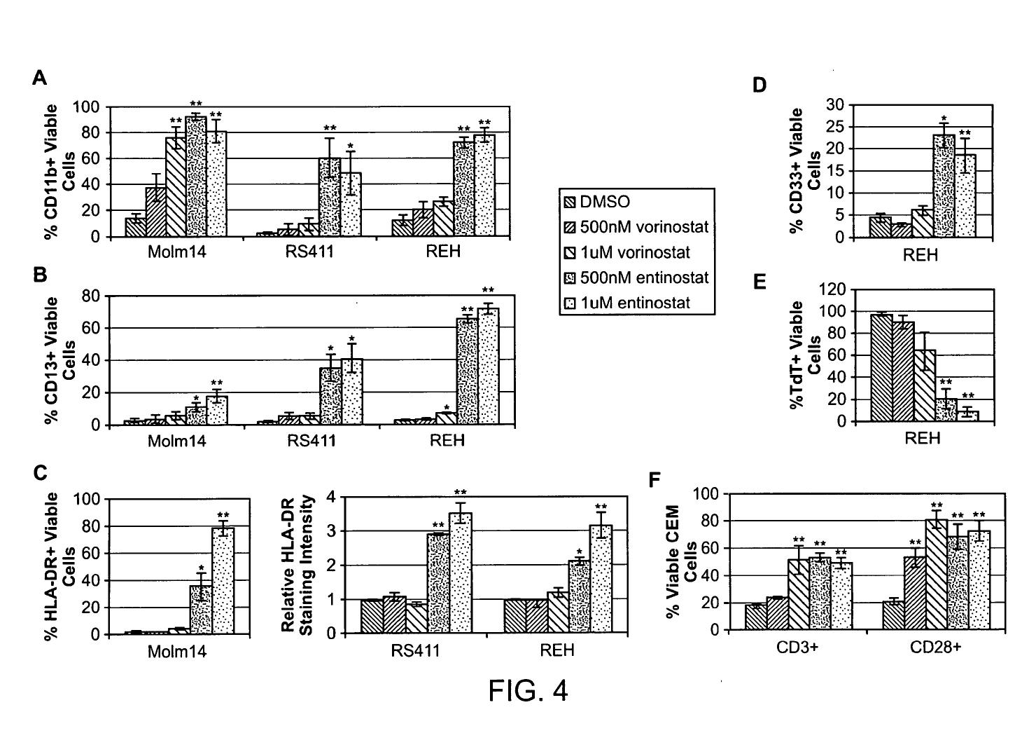

ALL cell lines (REH and RS411) and an AML cell line (Molm14) were treated with vorinostat (SAHA), entinostat (SNDX-275), or vehicle only for 72 hours. Cell surface or cytoplasmic expression of differentiation markers was determined by flow cytometric analysis of cells stained with fluorochrome-labeled antibodies (FIGS. 4 and 5C).

### Example 8

### Assessment of Interactions Between Histone Deacetylase Inhibitors (HDACIs)

ALL cell lines (REH and RS411) and an AML cell line (Molm14) were treated with vorinostat (SAHA), entinostat (SNDX-275), or both. Tumor cells were treated concurrently with both HDACIs for 48 hours (FIGS. 6 and 7) or were treated with one HDACI for 24 hours, after which the first HDACI was removed and the cells were cultured with the second HDACI for an additional 48 hours (FIG. 7). Relative cell numbers were determined by MTT assay and median effect analysis was used to determine Combination Index (CI) values. CI values were calculated using the equation CI=CA/ECA+CB/ECB, where CA and CB are the concentrations of the drugs A and B in the combination which elicit a certain effect and ECA and ECB are the isoeffective concentrations of the drugs A and B as single agents. CI Values less than 1 indicate synergistic anti-tumor activity.

What is claimed is:

1. A method for treating cancer in a subject in need thereof, said method comprising administering to said patient a first amount of a Class I selective HDAC inhibitor and a second amount of a second HDAC inhibitor.

2. The method of claim 1, wherein said first amount of said Class I selective HDAC inhibitor and said second amount of said second HDAC inhibitor are together an effective amount to provide a synergistic therapeutic anti-cancer effect.

3. The method of one of claim 1 or 2, wherein said first amount is a therapeutically effective amount and said second amount is a therapeutically effective amount.

4. The method of claim 1, wherein the second amount of the second HDAC inhibitor is less than a therapeutic amount of the second HDAC inhibitor when the second HDAC inhibitor is administered without the Class I selective HDAC inhibitor.

5. The method of claim 1, wherein the first amount of the Class I selective HDAC inhibitor is less than a therapeutic amount of the Class I selective HDAC when the Class I selective HDAC inhibitor is administered without the second HDAC inhibitor.

6. The method of claim 1, wherein the Class I selective HDAC inhibitor forces G₁ arrest.

7. The method of claim 1, wherein the Class I selective HDAC inhibitor is selected from entinostat, sodium phenyl butyrate, MGCD-0103, FK228, spiruchostatin A, SK7041, SK7068 or a 6-amino nicotinamide.

8. The method of claim 5, wherein the Class I selective HDAC inhibitor is entinostat.

9. The method of claim 1, wherein the second HDAC inhibitor forces G₂ arrest.

10. The method of claim 1 wherein the second HDAC inhibitor is a non-selective HDAC inhibitor.

11. The method of claim 1, wherein the second HDAC inhibitor is vorinostat, pyroxamide, CBHA, trichostatin A, trichostatin C, salicylihydroxamic acid, azelaic bihydroxamic acid, azelaic-1-hydroxamate-9-analide, 6-(3-chlorophenylureido) carpoic hydroxamic acid (3Cl-UCHA), oxamflatin, A-161906, scriptaid, PXD-101, LAQ-824, CHAP, MW2796, LBH589 or MW2996.

12. The method of claim 1, wherein the second HDAC inhibitor is vorinostat or trichostatin A.

13. The method of claim 10, wherein the second HDAC inhibitor is vorinostat.

14. The method of claim 1, further comprising administering an additional cancer therapy to the patient.

15. The method of claim 12 wherein the additional cancer therapy is selected from surgery, radiation therapy, or at least one chemotherapeutic agent.

16. The method of claim 13, wherein the chemotherapeutic is selected from anticancer agents, alkylating agents, cytotoxic agents, antimetabolic agents, hormonal agents, plant-derived agents, and biologic agents.

17. The method of claim 15, wherein the chemotherapeutic agent is selected from adriamycin, gemcitabine, mitomycin C, cisplatin, carboplatin, oxaliplatin, fluorouracil, leucovorin, cytarabine, etoposide, capecitabine, temozolomide, doxorubicin, daunomycin, daunorubicin, paclitaxel, docetaxel, cyclophosphamide, ifosfamide, methotrexate, clofarabine, forodesine, bevacizumab, and trastuzumab.

18. The method of claim 1, further comprising administering a methyltransferase inhibitor to the patient.

19. The method of claim 17, wherein the methyltransferase inhibitor is 5-azacytidine, decitabine or 5-aza-2′-deoxy-cytidine.

20. The method of claim 1, further comprising administering a proteasome inhibitor to the patient.

21. The method of claim 19, wherein the proteasome inhibitor is bortezomib, PR-171, salinosporamide A (NPI-0052), MG-132, omuralide, lactacystin or NEOSH101.

22. The method of claim 1, further comprising administering a kinase inhibitor.

23. The method of claim 21, wherein the kinase inhibitor is a dual kinase inhibitor.

24. The method of claim 21, wherein the kinase inhibitor is gefitinib, erlotinib, PKI-166, RPI.4610 (Angiozyme), sorafenib, AE-941 (Neovastat), OSI-774 (Tarceva®), PTK787, bevacizumab, trastuzumab, imatinib mesylate, ZD1839 (Iressa) and cetuximab (Erbitux®).

25. The method of claim 1, wherein the Class I selective HDAC inhibitor and the second HDAC inhibitor are co-administered.

26. The method of claim 1, wherein the cancer is brain cancer, breast cancer, lung cancer, ovarian cancer, pancreatic cancer, prostate cancer, renal cancer, colorectal cancer, leukemia, myeloid leukemia, glioblastoma, follicular lymphoma, pre-B acute leukemia, chronic lymphocytic B-leukemia, mesothelioma or small cell line cancer.

27. A pharmaceutical composition for treating cancer comprising a first amount of a Class I selective HDAC inhibitor and a second amount of a second HDAC inhibitor.

28. The pharmaceutical composition of claim 27, wherein said first amount and said second amount are together an effective amount to provide a synergistic therapeutic anti-cancer effect.

29. The pharmaceutical composition of claim 27, wherein the second amount of the second HDAC inhibitor is less than a therapeutic amount of the second HDAC inhibitor when the second HDAC inhibitor is administered without the Class I selective HDAC inhibitor.

30. The pharmaceutical composition claim 27, wherein the first amount of the Class I selective HDAC inhibitor is less than a therapeutic amount of the Class I selective HDAC when the Class I selective HDAC inhibitor is administered without the second HDAC inhibitor.

31. The pharmaceutical composition of claim 27, wherein the pharmaceutical composition is an oral dosage form comprising a prolonged release portion, wherein said prolonged release portion comprises said second HDAC inhibitor.

32. The pharmaceutical composition of one of claims 27 to 31, wherein said Class I selective HDAC inhibitor is entinostat, sodium phenyl butyrate, MGCD-0103, FK228, spiruchostatin A, SK7041, SK7068 or a 6-amino nicotinamide.

33. The pharmaceutical composition of one of claims 27 to 31, wherein said Class I selective HDAC inhibitor is entinostat.

34. The pharmaceutical composition of one of claims 27 to 31, wherein said second HDAC inhibitor is vorinostat, pyroxamide, CBHA, trichostatin A, trichostatin C, salicylihydroxamic acid, azelaic bihydroxamic acid, azelaic-1-hydroxamate-9-analide, 6-(3-chlorophenylureido) carpoic hydroxamic acid (3Cl-UCHA), oxamflatin, A-161906, scriptaid, PXD-101, LAQ-824, CHAP, MW2796, LBH589 or MW2996.

35. The pharmaceutical composition of one of claims 27 to 31, wherein said second HDAC inhibitor is vorinostat or trichostatin C.

36. The pharmaceutical composition of one of claims 27 to 31, wherein said second HDAC inhibitor is vorinostat.

37. A kit for treating cancer comprising a first pharmaceutical composition comprising a first amount of a Class I selective HDAC inhibitor and a second pharmaceutical composition comprising a second amount of a second HDAC inhibitor.

38. The kit of claim 37, wherein said second pharmaceutical composition is an oral dosage form comprising a prolonged release portion, wherein said prolonged release portion comprises said second HDAC inhibitor.

39. The kit of claim 37, wherein said first amount and said second amount are together an effective amount to provide a synergistic therapeutic anti-cancer effect.

40. The kit of claim 37, wherein the second amount of the second HDAC inhibitor is less than a therapeutic amount of the second HDAC inhibitor when the second HDAC inhibitor is administered without the Class I selective HDAC inhibitor.

41. The kit of claim 37, wherein the first amount of the Class I selective HDAC inhibitor is less than a therapeutic amount of the Class I selective HDAC when the Class I selective HDAC inhibitor is administered without the second HDAC inhibitor.

42. The kit of claim 37, wherein the first pharmaceutical composition and the second pharmaceutical composition are independently selected from an injectable dosage form and a oral dosage form.

43. The kit of claim 37, wherein the first pharmaceutical composition and the second pharmaceutical composition are injectable dosage forms.

44. The kit of one of claims 37 to 43, wherein said Class I selective HDAC inhibitor is entinostat, sodium phenyl butyrate, MGCD-0103, FK228, spiruchostatin A, SK7041, SK7068 or a 6-amino nicotinamide.

45. The kit of one of claims 37 to 43, wherein said Class I selective HDAC inhibitor is entinostat.

46. The kit of one of claims 37 to 43, wherein said second HDAC inhibitor is vorinostat, pyroxamide, CBHA, trichostatin A, trichostatin C, salicylihydroxamic acid, azelaic bihydroxamic acid, azelaic-1-hydroxamate-9-analide, 6-(3-chlorophenylureido) carpoic hydroxamic acid (3Cl-UCHA), oxamflatin, A-161906, scriptaid, PXD-101, LAQ-824, CHAP, MW2796, LBH589 or MW2996.

47. The kit of one of claims 37 to 43, wherein said second HDAC inhibitor is vorinostat or trichostatin C.

48. The kit of one of claims 37 to 43, wherein said second HDAC inhibitor is vorinostat.
